(19) United States
(12) Patent Application Publication
(10) Pub. No.: US 2021/0214434 A1
(43) Pub. Date: Jul. 15, 2021

(54) VARIANTS WITH FC FRAGMENT HAVING AN INCREASED AFFINITY FOR FCRN AND AN INCREASED AFFINITY FOR AT LEAST ONE RECEPTOR OF THE FC FRAGMENT

(71) Applicant: LABORATOIRE FRANÇAIS DU FRACTIONNEMENT ET DES BIOTECHNOLOGIES, LES ULIS (FR)

(72) Inventors: Harry MEADE, Newton, MA (US); Céline MONNET, LAMBERSART (FR); Philippe MONDON, DONNEVILLE (FR)

(21) Appl. No.: 16/772,244

(22) PCT Filed: Dec. 14, 2018
(86) PCT No.: PCT/EP2018/084970

§ 371 Date: Jun. 12, 2020

(30) Foreign Application Priority Data
Dec. 15, 2017 (FR) 1762217

(51) Int. Cl. C07K 16/28; A61P 19/02; A61P 37/06; C07K 16/04; A61P 7/00

(52) U.S. Cl. CPC C07K 16/283; A61P 19/02; A61P 37/06; A61K 2039/505; A61P 7/00; C07K 2317/92; C07K 16/04

(57) ABSTRACT

Disclosed is a variant of a parent polypeptide including an Fc fragment, the variant having an increased affinity for the FcRn receptor, and an increased affinity for at least one receptor of the Fc fragment (FcR) chosen from the FcγRI (CD64), FcγRIIIa (CD16a) and FcγRIIa (CD32a) receptors, relative to that of the parent polypeptide, characterised in that it includes: (i) the four mutations 334N, 352S, 378V and 397M; and (ii) at least one mutation chosen from 434Y, 434S, 226G, P228L, P228R, 230S, 230T, 230L, 241L, 264E, 307P, 315D, 330V, 362R, 389T and 389K; the numbering being that of the EU index or the Kabat equivalent.

### BACKGROUND OF THE INVENTION

### Field of the Invention

The present invention relates to a polypeptide (also called variant) comprising a mutated Fc region and having increased affinity for the FcRn receptor, as well as increased affinity for at least one Fc receptor (FcR) relative to a parent polypeptide.

### Description of the Related Art

An antibody consists of a tetramer of heavy and light chains. The two light chains are identical to each other, while the two heavy chains are identical and connected by disulfide bridges. There are five types of heavy chains (alpha, gamma, delta, epsilon, mu), which determine immunoglobulin classes (IgA, IgG, IgD, IgE, IgM). The light chain group includes two subtypes, lambda and kappa.

IgGs are soluble antibodies that may be found in blood and other body fluids. IgG is a Y-shaped glycoprotein with an approximate molecular weight of 150 kDa, consisting of two heavy and two light chains. Each chain stands out by a constant region and a variable region. The two carboxy-terminal domains of the heavy chains form the Fc fragment, while the amino-terminal domains of the heavy and light chains recognize the antigen and are called the Fab fragment.

The Fc fusion proteins are created by a combination of an antibody Fc fragment with a protein domain that provides the specificity for a given therapeutic target. Examples are combinations of the Fc fragment with any type of therapeutic proteins or fragments thereof.

Fc polypeptides, in particular Fc fragments, therapeutic antibodies and Fc fusion proteins, are used today to treat various diseases, such as rheumatoid arthritis, psoriasis, multiple sclerosis and many forms of cancer. Therapeutic antibodies may be monoclonal or polyclonal antibodies. The monoclonal antibodies are obtained from a single antibody-producing cell line, which shows identical specificity for a single antigen. The therapeutic Fc fusion proteins are used or developed as drugs against autoimmune diseases and/or inflammatory component, such as etanercept (Amgen's Enbrel, which is an Fc-bound TNF receptor) or Alefacept (Biogen Idec's Amevive, which is LFA-3 bound to the Fc portion of human IgG1).

Fc polypeptides, such as the Fc fragments, Fc antibodies and fusion proteins, have, in particular, an activity dependent on the binding of their Fc part to their receptors, i.e. FcRn and the Fc fragment receptors (FcR), such as FcγRI (CD64), FcγRIIIa (CD16a) and FcγRIIa (CD32a) receptors.

One of the desired effects in therapies involving Fc polypeptide interactions with Fc fragment receptors (FcR) is inhibition of immune system activation by binding to Fc receptors on the surface of effector cells. Particularly in the context of the treatment of inflammatory and/or autoimmune diseases, involving autoantibodies and/or cytokines, Fc-based therapies can act by blocking Fc receptors and thus by competing with autoantibodies for access to these receptors. This results in inhibition of direct activities normally mediated by autoantibodies (e.g. antibody-dependent cellular cytotoxicity, complement-dependent cytotoxicity, or antibody-dependent cellular phagocytosis), and decreased activation of the immune system, including cytokine release. In addition, since the FcRn receptor is involved in the recycling of antibodies, blocking them with Fc polypeptides allows faster elimination of autoantibodies, thus reducing their half-life. This is why treatments based on Fc fragments are particularly suitable for autoimmune and/or inflammatory diseases, triggered by uncontrolled stimulation of the cells of the immune system, in particular by autoantibodies and/or cytokines.

The basic therapy proposed for the treatment of these diseases is an intravenous immunoglobulin (IVIG or IVIg) therapy which consists in intravenously administering to the patients immunoglobulins (IgG most often) from pools of human plasma donations. It is generally accepted that these IgGs act, in particular, by blocking the Fc receptors and thus competing with the autoantibodies for access to these receptors. More recently, Fc fragments have been developed for the purpose of modifying their Fc receptor binding properties. Nevertheless, their effectiveness remains to be demonstrated.

There is still a need to optimize these Fc fragments, in particular to increase their half-life, and/or their therapeutic efficacy.

The Applicant has now developed particular Fc fragments exhibiting improved activity, in particular by an improved FcRn binding affinity. These Fc fragments may be used in therapy, and are particularly suitable for the treatment of inflammatory and/or autoimmune diseases, in order to bring greater effectiveness to the product that contains them.

In particular, these fragments may exhibit a more efficient blockade of Fc receptors present on the cells of the immune system, which are then less, or no longer, accessible for the binding of autoantibodies, whose activity is then inhibited.

In addition, Fc fragments make it possible to block the FcRn receptor more efficiently and thus eliminate autoantibodies more quickly.

In addition, some of these particular Fc fragments have, as demonstrated in the examples, better inhibition of complement-dependent cytotoxicity (CDC) than IVIG. They therefore make it possible to reduce the toxicity of pathogenic autoantibodies, such as those involved in inflammatory and/or autoimmune diseases.

### SUMMARY OF THE INVENTION

The present invention thus provides a variant of a parent polypeptide having optimized properties relating to functional activity mediated by the Fc region.

The present invention thus relates to a variant of a parent polypeptide comprising an Fc fragment, said variant having an increased affinity for the FcRn receptor, and an increased affinity for at least one Fc receptor (FcR) selected from FcγRI receptors. (CD64), FcγRIIIa (CD16a) and FcγRIIa (CD32a), relative to that of the parent polypeptide, characterized in that it comprises:
(i) the four mutations 334N, 352S, 378V and 397M; and
(ii) at least one mutation selected from 434Y, 434S, 226G, P228L, P228R, 230S, 230T, 230L, 241L, 264E, 307P, 315D, 330V, 362R, 389T and 389K;

wherein the numbering is that of the EU index or equivalent in Kabat.

According to one embodiment, the variant according to the invention further comprises at least one mutation (iii) in the Fc fragment chosen from among Y296W, K290G, V240H, V240I, V240M, V240N, V240S, F241H, F241Y, L242A, L242F, L242G, L242H, L242I, L242K, L242P, L242S, L242T, L242V, F243L, F243S, E258G, E258I, E258R, E258M, E258Q, E258Y, V259C, V259I, V259L, T260A, T260H, T260I, T260M, T260N, T260R, T260S, T260W, V262S, V263T, V264L, V264S, V264T, V266L, S267A, S267Q, S267V, K290D, K290E, K290H, K290L, K290N, K290Q, K290R, K290S, K290Y, P291G, P291Q, P291R, R292I, R292L, E293A, E293D, E293G, E293M, E293Q, E293S, E293T, E294A, E294G, E294P, E294Q, E294R, E294T, E294V, 02951, Q295M, Y296H, S298A, S298R, Y300I, Y300V, Y300W, R301A, R301M, R301 P, R301 S, V302F, V302L, V302M, V302R, V302S, V303S, V303Y, 5304T, V305A, V305F, V3051, V305L, V305R and V305S,

wherein the numbering is that of the EU index or equivalent in Kabat.

Such a variant is called “variant according to the invention”, “mutant according to the invention” or “polypeptide according to the invention”.

Preferably, the variant according to the invention has both an increased affinity for the FcRn receptor and an increased affinity for all FcγRI (CD64), FcγRIIIa (CD16a) and FcγRIIa (CD32a) receptors.

Preferably, in addition, the variant according to the invention is capable of inhibiting complement-dependent cytotoxicity (CDC), attributed to a modification of binding to complement proteins, in particular C1q. This inhibition is significantly improved compared to that conferred by IVIG.

Preferably, the variant according to the invention is different from the variant consisting of an Fc fragment, in particular of IgG1, having the five mutations N434Y, K334N, P352S, V397M and A378V, and produced in HEK293 cells, wherein the numbering is that of the EU index or equivalent in Kabat. Thus, preferably, the variant according to the invention is different from the Fc fragment, in particular IgG1, N434Y/K334N/P352S/V397M/A378V produced in HEK293 cells, wherein the numbering is that of the EU index or equivalent in Kabat.

Throughout this application, the numbering of residues in the Fc region is that of the immunoglobulin heavy chain according to the EU index or equivalent in Kabat et al. (Sequences of Proteins of Immunological Interest, 5th ed., Public Health Service, National Institutes of Health, Bethesda, Md., 1991). The term “EU index or equivalent in Kabat” refers to the US numbering of the residues of the human IgG1, IgG2, IgG3 or IgG4 antibody. This is illustrated on the IMGT website (http://www.imat.ora/IMGTScientificChart/Numberina/HuIG HGnber.html).

By “polypeptide” or “protein” is meant a sequence comprising at least 100 covalently-attached amino acids.

By “amino acid” is meant one of the 20 naturally occurring amino acids or non-natural analogues.

The term “position” means a position in the sequence of a polypeptide. For the Fc region, the positions are numbered according to the EU index or equivalent in Kabat. The term “antibodies” is used in the everyday sense. It corresponds to a tetramer that comprises at least one Fc region, and two variable regions. Antibodies comprise, but are not limited to, full-length immunoglobulins, monoclonal antibodies, multi-specific antibodies, chimeric antibodies, humanized antibodies, and fully human antibodies. The amino-terminal portion of each heavy chain comprises a variable region of about 100 to 110 amino acids responsible for antigen recognition. In each variable region, three loops are pooled to form an antigen binding site. Each of the loops is called a complementarity determining region (hereinafter referred to as a “CDR”). The carboxy terminal portion of each heavy chain defines a constant region that is primarily responsible for the effector function.

IgGs have several subclasses, in particular IgG1, IgG2, IgG3 and IgG4. The subclasses of IgM are, in particular, IgM1 and IgM2. Thus, by “isotype” is meant one of the subclasses of immunoglobulins defined by the chemical and antigenic characteristics of their constant regions. The known isotypes of human immunoglobulins are IgG1, IgG2, IgG3, IgG4, IgA1, IgA2, IgM1, IgM2, IgD and IgE.

Full length IgGs are tetramers and consist of two identical pairs of two immunoglobulin chains, each pair having a light chain and a heavy chain, wherein each light chain comprises the VL and CL domains, and each heavy chain comprises the domains VH, Cγ1 (also called CH1), Cγ2 (also called CH2), and Cy3 (also called CH3). In the context of a human IgG1, “CH1” refers to positions 118 to 215, “CH2” refers to positions 231 to 340, and “CH3” refers to positions 341 to 447 according to the EU index or equivalent in Kabat. The IgG heavy chain also includes an N-terminal flexible hinge domain which refers to positions 216-230 in the case of IgG1. The lower hinge range refers to positions 226 to 230 according to the EU index or equivalent in Kabat.

By “variable region” is meant the region of an immunoglobulin which comprises one or more Ig domains substantially encoded by any of the VK, Vλ and/or VH genes that make up the kappa, lambda, and immunoglobulin heavy chains, respectively. Variable regions include complementarity determining regions (CDRs) and framework regions (FRs).

The term “Fc” or “Fc region” refers to the constant region of an antibody excluding the first domain of the immunoglobulin constant region (CH1). Thus Fc refers to the last two domains (CH2 and CH3) of the IgG1 constant region, and to the flexible N-terminal hinge of these domains. For a human IgG1, the Fc region corresponds to the residue C226 at its carboxy terminal end, i.e. the residues of the position 226 to 447, where the numbering is according to the EU index or equivalent in Kabat. The Fc region used may further comprise a portion of the upper hinge region located between positions 216-226 according to the EU index or equivalent in Kabat; in this case, the Fc region used corresponds to the residues of the position 216 to 447, 217 to 447, 218 to 447, 219 to 447, 220 to 447, 221 to 447, 222 to 447, 223 to 447, 224 to 447 or 225 to 447, wherein the numbering is according to the EU index or equivalent in Kabat. Preferably in this case, the Fc region used corresponds to the residues of position 216 to 447, wherein the numbering is according to the EU index or equivalent in Kabat.

Preferably, the Fc region used is chosen from the sequences SEQ ID NO: 1 to 10 and 14.

By “parent polypeptide” is meant a reference polypeptide. The said parent polypeptide may be of natural or synthetic origin. In the context of the present invention, the parent polypeptide comprises an Fc region, referred to as the “parent Fc region”. This Fc region may be selected from the group of wild-type Fc regions, their fragments and mutants. Preferably, the parent polypeptide comprises a human Fc fragment, preferably an Fc fragment of a human IgG1 or a human IgG2. The parent polypeptide may include preexisting amino acid modifications in the Fc region (e.g. Fc mutant) relative to wild-type Fc regions.

Advantageously, the parent polypeptide may be an isolated Fc region (i.e. an Fc fragment as such), a sequence derived from an isolated Fc region, an antibody, an antibody fragment comprising an Fc region, a fusion protein comprising an Fc region or a conjugate Fc, wherein this list is not limiting.

By “sequence derived from an isolated region Fc” is meant a sequence comprising at least two isolated Fc regions linked together, such as an scFc (single chain Fc) or a multimer Fc. By “fusion protein comprising an Fc region” is meant a polypeptide sequence fused to an Fc region, said polypeptide sequence being preferably selected from variable regions of any antibody, sequences binding a receptor to its ligand, adhesion molecules, ligands, enzymes, cytokines and chemokines. By “Fc conjugate” is meant a compound that is the result of the chemical coupling of an Fc region with a conjugation partner. The conjugation partner may be protein or non-protein. The coupling reaction generally utilizes functional groups on the Fc region and the conjugation partner. Various binding groups are known in the prior art as being suitable for the synthesis of a conjugate; for example, homo- or heterobifunctional binders are well known (see, Pierce Chemical Company catalog, 2005-2006, technical section on crosslinking agents, pages 321-350). Suitable conjugation partners include therapeutic proteins, labels, cytotoxic agents such as chemotherapeutic agents, toxins and their active fragments. Suitable toxins and fragments thereof include diphtheria toxin, exotoxin A, ricin, abrin, saporin, gelonin, calicheolyin, auristatin E and F, and mertansin.

Advantageously, the parent polypeptide—and therefore the polypeptide according to the invention—consists of an Fc region.

Advantageously, the parent polypeptide—and therefore the polypeptide according to the invention—is an antibody.

By “mutation” is meant a change of at least one amino acid of the sequence of a polypeptide, including a change of at least one amino acid of the Fc region of the parent polypeptide. The mutated polypeptide thus obtained is a variant polypeptide; it is a polypeptide according to the invention. Such a polypeptide comprises a mutated Fc region, relative to the parent polypeptide. Preferably, the mutation is a substitution, an insertion or a deletion of at least one amino acid.

By “substitution” is meant the replacement of an amino acid at a particular position in a parent polypeptide sequence by another amino acid. For example, the N434S substitution refers to a variant polypeptide, in this case a variant for which asparagine at position 434 is replaced by serine.

By “amino acid insertion” or “insertion” is meant the addition of an amino acid at a particular position in a parent polypeptide sequence. For example, insertion G>235-236 refers to a glycine insertion between positions 235 and 236.

By “amino acid deletion” or “deletion” is meant the deletion of an amino acid at a particular position in a parent polypeptide sequence. For example, E294del refers to the removal of glutamic acid at position 294.

Preferably, the following mutation label is used: “434S” or “N434S”, and means that the parent polypeptide comprises asparagine at position 434, which is replaced by serine in the variant. In the case of a combination of substitutions, the preferred format is “259I/315D/434Y” or “V259I/N315D/N434Y”. This means that there are three substitutions in the variant, at positions 259, 315 and 434, and that the amino acid at position 259 of the parent polypeptide, i.e. valine, is replaced by isoleucine, that the amino acid at position 315 of the parent polypeptide, asparagine, is replaced by aspartic acid, and that the amino acid at position 434 of the parent polypeptide, asparagine, is replaced by tyrosine.

By “FcRn” or “neonatal Fc receptor” as used herein is meant a protein that binds to the Fc region of IgG and is encoded at least in part by an FcRn gene. As is known in the prior art, the functional FcRn protein comprises two polypeptides, often referred to as heavy chain and light chain. The light chain is beta-2-microglobulin, while the heavy chain is encoded by the FcRn gene. Unless otherwise noted herein, FcRn or FcRn protein refers to the α-chain complex with beta-2-microglobulin. In humans, the gene encoding FcRn is called FCGRT.

Preferably, the variant according to the invention has an increased affinity for the FcRn receptor, relative to that of the parent polypeptide, by a ratio at least equal to 2, preferably greater than 5, more preferably greater than 10, even more preferably greater than 15, particularly preferably greater than 20, even more particularly preferably greater than 25, most preferably greater than 30.

Preferably, the variant according to the invention has an increased half-life compared to that of the parent polypeptide. Preferably, the variant according to the invention has an increased half-life with respect to that of the parent polypeptide, by a ratio at least equal to 2, preferably greater than 5, more preferably greater than 10, even more preferably greater than 15, particularly preferably greater than 20, even more particularly preferably greater than 25, most preferably greater than 30.

One of the major functions of FcRn is known as IgG recycling. It consists of extracting IgG from the endothelial catabolism pathway of plasma proteins to restore them intact to the circulation. This recycling explains their half-life under normal physiological conditions (three weeks for IgG), while maintaining high plasma concentrations. The transcytosis of IgG from one pole to the other of epithelia or endothelium is the second major function of FcRn to ensure their biodistribution in the body.

Preferably, the variant according to the invention has an increased affinity for at least one receptor of the Fc fragment (FcR) chosen from the receptors FcγRI (CD64), FcγRIIIa (CD16a) and FcγRIIa (CD32a), with respect to that of the parent polypeptide, by a ratio at least equal to 2, preferably greater than 5, more preferably greater than 10, even more preferably greater than 15, particularly preferably greater than 20, even more particularly preferably greater than 25, most preferably greater than 30.

The FcγRI receptor (CD64) is involved in phagocytosis and cell activation. The FcγRIIIa receptor (CD16a) is also involved in Fc-dependent activity, including ADCC and phagocytosis; it has a V/F polymorphism at position 158. The FcγRIIa receptor (CD32a) is, in turn, involved in platelet activation and phagocytosis; it has an H/R polymorphism at position 131.

Preferably, the variant according to the invention has both an increased affinity for the FcRn receptor, and an increased affinity for all FcγRI (CD64), FcγRIIIa (CD16a) and FcγRIIa (CD32α) receptors.

The affinity of a polypeptide comprising an Fc region for an FcR may be evaluated by methods well known in the prior art. For example, those skilled in the art may determine the affinity (Kd) using surface plasmon resonance (SPR). Alternatively, those skilled in the art may perform an appropriate ELISA test. An appropriate ELISA assay compares the binding forces of the parent Fc and the mutated Fc. The specific detected signals for the mutated Fc and the parent Fc are compared. Binding affinity may be indifferently determined by evaluating whole polypeptides or evaluating isolated Fc regions thereof. Alternatively, those skilled in the art may perform an appropriate competitive assay. An appropriate competitive assay is used to determine the ability of the mutated Fc to inhibit the binding of a labeled FcR ligand when these are incubated simultaneously with cells expressing these receptors. The binding of the labeled ligand to FcR may be evaluated, for example, by flow cytometry. The binding affinity of the Fc mutated at FcR is then determined by evaluating the variability of the average fluorescence intensity emitted by the labeled ligand bound to the FcR.

Preferably, the mutated Fc region of the polypeptide according to the invention comprises from 3 to 20 mutations relative to the parent polypeptide, preferably from 4 to 20 mutations. By “from 3 to 20 amino acid modifications” is meant 3, 4, 5, 6, 7, 8, 9, 10, 11, 12, 13, 14, 15, 16, 17, 18, 19 and 20 amino acid mutations. Preferably, it comprises from 4 to 15 mutations, more preferably from 4 to 10 mutations relative to the parent polypeptide.

Even more preferably, the mutated Fc region of the polypeptide according to the invention may comprise at least one combination of 5 mutations, said combination comprising the four mutations (i) as described above, and at least one mutation (ii) as described above, wherein the numbering is that of the EU index or equivalent in Kabat.

Even more preferably, the mutated Fc region of the polypeptide according to the invention comprises a combination of 6 mutations, said combination comprising the four mutations (i) as described above, at least one mutation (ii) as described above, and at least one mutation (iii) as described above, wherein the numbering is that of the EU index or equivalent in Kabat.

Preferably, the mutated Fc region of the polypeptide according to the invention comprises the following mutations:
(i) the four mutations 334N, 352S, 378V and 397M;
(ii) at least one mutation selected from 434Y, 434S, 226G, P228L, P228R, 230S, 230T, 230L, 241L, 264E, 307P, 315D, 330V, 362R, 389T and 389K; and
when a mutation (iii) is present, it is selected from K290G and Y296W, wherein the numbering is that of the EU index or equivalent in Kabat.

Preferably, the mutated Fc region of the polypeptide according to the invention comprises the following mutations:
(i) the four mutations 334N, 352S, 378V and 397M;
(ii) at least one mutation selected from 434Y, 434S, 226G, P228L, P228R, 230S, 230T, 230L, 241L, 264E, 307P, 315D, 330V, 362R, 389T and 389K; and
(iii) at least one mutation selected from K290G and Y296W, wherein the numbering is that of the EU index or equivalent in Kabat.

Preferably, the mutated Fc region of the polypeptide according to the invention comprises a combination of mutations chosen from the combinations: N434Y/K334N/P352S/V397M/A378V and N434Y/K334N/P352S/V397M/A378V/Y296W.

Preferably, the polypeptide according to the invention is produced in mammary epithelial cells of transgenic non-human mammals.

Preferably, the polypeptide according to the invention is produced in non-human transgenic animals, preferably in transgenic non-human mammals, more preferably in their mammary epithelial cells.

By “transgenic non-human mammal” is meant a mammal chosen, in particular, from among cattle, pigs, goats, sheep and rodents, preferably from among the goat, the mouse, the sow, the rabbit, the ewe and the cow. Preferably, the transgenic non-human animal or the transgenic non-human mammal is a transgenic goat.

Preferably, the variant according to the invention comprises at least the five mutations N434Y, K334N, P352S, V397M and A378V in its Fc fragment, and is produced in mammary epithelial cells of transgenic non-human mammals, or in transgenic non-human animals, preferably in transgenic non-human mammals, such as a goat. Such a variant has both increased affinity for the FcRn receptor, and increased affinity for all FcγRI (CD64), FcγRIIIa (CD16a) and FcγRIIa (CD32a) receptors.

Thus, preferably, the variant according to the invention is the Fc N434Y/K334N/P352S/V397M/A378V variant produced in mammary epithelial cells of transgenic non-human mammals. Alternatively, preferably, the variant according to the invention is the Fc N434Y/K334N/P352S/V397M/A378V variant produced in transgenic non-human animals, preferably in transgenic non-human mammals, such as a goat. Such a variant has both increased affinity for the FcRn receptor, and increased affinity for all FcγRI (CD64), FcγRIIIa (CD16a) and FcγRIIa (CD32a) receptors. Preferably, the variant according to the invention comprises the sequence SEQ ID NO: 11 or the sequence SEQ ID NO: 15.

Alternatively, preferably, the variant according to the invention is the variant Fc N434Y/K334N/P352S/V397M/A378V/Y296W produced in mammary epithelial cells of transgenic non-human mammals. Alternatively, preferably, the variant according to the invention is the Fc N434Y/K334N/P352S/V397M/A378V/Y296W variant produced in transgenic non-human animals, preferably in transgenic non-human mammals, such as a goat. Such a variant has both increased affinity for the FcRn receptor, and increased affinity for all FcγRI (CD64), FcγRIIIa (CD16a) and FcγRIIa (CD32a) receptors.

Preferably, the method for producing a variant according to the invention comprises the expression of said variant in mammary epithelial cells of transgenic non-human mammals.

Thus, the present invention also relates to a method for producing a variant of a parent polypeptide comprising an Fc fragment, said variant having an increased affinity for the FcRn receptor, and an increased affinity for at least one Fc receptor (FcR) selected from FcγRI (CD64), FcγRIIIa (CD16a) and FcγRIIa (CD32a) receptors, relative to that of the parent polypeptide, said variant comprising:
(i) the four mutations 334N, 352S, 378V and 397M; and
(ii) at least one mutation selected from 434Y, 434S, 226G, P228L, P228R, 230S, 230T, 230L, 241L, 264E, 307P, 315D, 330V, 362R, 389T and 389K; and
wherein the numbering is that of the EU index or equivalent in Kabat, said method comprising expressing said variant in mammary epithelial cells of transgenic non-human mammals.

Preferably, said variant further comprises at least one mutation (iii) in the Fc fragment chosen from among Y296W, K290G, V240H, V240I, V240M, V240N, V240S, F241H, F241Y, L242A, L242F, L242G, L242H, L242I, L242K, L242P, L242S, L242T, L242V, F243L, F243S, E258G, E258I, E258R, E258M, E258Q, E258Y, V259C, V259I, V259L, T260A, T260H, T260I, T260M, T260N, T260R, T260S, T260W, V262S, V263T, V264L, V264S, V264T, V266L, S267A, S267Q, S267V, K290D, K290E, K290H, K290L, K290N, K290Q, K290R, K290S, K290Y, P291G, P291Q, P291R, R292I, R292L, E293A, E293D, E293G, E293M, E293A, E293S, E293T, E294A, E294G, E294P, E294Q, E294R, E294T, E294 Q295I, Q295M, Y296H, S298A, S298R, Y300I, Y300V, Y300W, R301A, R301M, R301P, R301S, V302F, V302L, V302M, V302R, V302S, V303S, V303Y, 5304T, V305A, V305F, V3051, V305L, V305R and V305S, wherein the numbering is that of the EU index or equivalent in Kabat.

In particular, such a method comprises the following steps:
a) preparing a DNA sequence comprising a sequence encoding the variant, a sequence encoding a mammalian casein promoter or a mammalian whey promoter, and a sequence encoding a signal peptide permitting the secretion of said variant;
b) introducing the DNA sequence obtained in a), into a non-human mammalian embryo, to obtain a transgenic non-human mammal expressing the variant encoded by said DNA sequence obtained in a) in the mammary gland; and
c) recovery of the variant in the milk produced by the transgenic non-human mammal obtained in b).

Step a) thus comprises the preparation of a DNA sequence comprising a sequence coding for the variant, a sequence coding for a mammalian casein promoter or a mammalian whey promoter, and a sequence coding for a signal peptide. allowing the secretion of said variant. Such a step is illustrated in FIG. 1.

The sequence coding for the variant is a DNA sequence coding for the variant according to the invention.

For example, this variant has the sequence SEQ ID NO: 11. With the signal peptide, the corresponding sequence is the sequence SEQ ID NO: 13.

In another example, this variant has the sequence SEQ ID NO: 15. With the signal peptide, the corresponding sequence is the sequence SEQ ID NO: 16.

The coding sequence for a mammalian casein promoter or a mammalian whey promoter makes it possible to express the variant in the milk. Those skilled in the art know how to choose such a promoter.

In the context of the present application, a signal peptide is an amino acid sequence, preferably from 2 to 30 amino acids, located at the N-terminus of the Fc polypeptide variant, serving to address it in the mammalian milk. Preferably, the coding sequence for a signal peptide is interposed between the sequence coding for the variant and the promoter. Without such a sequence, the variant would remain in the mammary tissue, wherein purification would be difficult and would require the sacrifice of the host animal. The signal peptide may be cleaved upon secretion. The coding sequence for the peptide signal may be one that is naturally associated with a parent polypeptide according to the invention. Alternatively, the coding sequence for the signal peptide may be that of the milk protein from which the promoter is derived, i.e. when the milk protein gene is digested in order to isolate the promoter, a DNA fragment is selected comprising both the promoter and the coding sequence of the signal peptide directly downstream of the promoter. Another alternative is to use a signal sequence derived from another secreted protein that is neither the milk protein normally expressed from the promoter, nor a polypeptide according to the invention.

Preferably, the signal peptide has the sequence SEQ ID NO: 12.

The DNA sequence used may comprise optimized codons.

Codon optimization aims to replace natural codons by codons whose transfer RNA (tRNA) carrying the amino acids are most common in the cell type in question. The mobilization of frequently encountered tRNAs has the major advantage of increasing the translation speed of messenger RNAs (mRNAs) and therefore of increasing the final titre (Carton, J. M. et al., Protein Expr Purif, 2007). Sequence optimization also plays on the prediction of mRNA secondary structures that could slow down reading by the ribosomal complex. Sequence optimization also has an impact on the percentage of G/C that is directly related to the half-life of the mRNAs and therefore to their potential to be translated (Chechetkin, J. of Theoretical Biology 242, 2006 922-934).

Codon optimization may be effected by substitution of natural codons using codon frequency tables (Codon Usage Table) for mammals and more specifically for Homo sapiens. There are algorithms available on the internet and made available by the suppliers of synthetic genes (DNA2.0, GeneArt, MWG, Genscript) that make this sequence optimization possible.

Preferably, step a) comprises the following steps:

(a1) preparing a DNA sequence comprising a sequence coding for the variant according to the invention, directly fused at its N-terminus to a sequence coding for a signal peptide allowing the secretion of said variant;

(a2) introducing the DNA sequence obtained in (a1) into a vector comprising a sequence coding for a mammalian casein promoter or a mammalian whey promoter;

(a3) digesting said vector obtained in (a2), in order to obtain a DNA sequence comprising the sequence coding for a mammalian casein promoter or a mammalian whey promoter, and the DNA sequence comprising a sequence encoding the variant of the invention directly fused at its N-terminus to a coding sequence for a signal peptide.

In other words, preferably, at the end of step a), we obtain a DNA sequence comprising, from the N- to C-terminus, the coding sequence for a mammalian casein promoter or a mammalian whey promoter, fused to the coding sequence for a signal peptide, itself fused to the coding sequence for the variant according to the invention.

Next, the process according to the invention comprises a step b) of introducing the DNA sequence obtained in a) into a non-human mammalian embryo, in order to obtain a transgenic non-human mammal expressing the variant coded by said sequence of DNA obtained in a) in the mammary gland.

Finally, the process according to the invention comprises a step c) of recovering the variant in the milk produced by the transgenic nonhuman mammal obtained in b).

Steps b) and c) are known from the prior art, in particular patent EP0264166.

Preferably, such a process comprises, after step c), a purification step d) of the recovered milk. The purification step d) may be carried out by any known process of the prior art, in particular by purification on protein A. Once again, such a step is described, in particular, in patent EP0264166.

The present invention also relates to a DNA sequence comprising a gene encoding a variant of a parent polypeptide comprising an Fc fragment, said variant having an increased affinity for the FcRn receptor, and an increased affinity for at least one fragment receptor Fc (FcR) chosen from among FcγRI (CD64), FcγRIIIa (CD16a) and FcγRIIa (CD32a) receptors, relative to that of the parent polypeptide, said variant comprising:
(i) the four mutations 334N, 352S, 378V and 397M; and
(ii) at least one mutation selected from 434Y, 434S, 226G, P228L, P228R, 230S, 230T, 230L, 241L, 264E, 307P, 315D, 330V, 362R, 389T and 389K;
wherein the numbering is that of the EU index or equivalent in Kabat,
said gene being under the control of a transcriptional promoter of mammalian casein or whey which does not naturally control the transcription of said gene, said DNA sequence further comprising a sequence coding for a signal peptide allowing secretion of said variant interposed between the sequence encoding the variant and the promoter.

In a particular embodiment, said variant further comprises at least one mutation (iii) in the Fc fragment chosen from among Y296W, K290G, V240H, V240I, V240M, V240N, V240S, F241H, F241Y, L242A, L242F, L242G, L242H, L242I, L242K, L242P, L242S, L242T, L242V, F243L, F243S, E258G, E258I, E258R, E258M, E258Q, E258Y, V259C, V259I, V259L, T260A, T260H, T260I, T260M, T260N, T260R, T260S, T260W, V262S, V263T, V264L, V264S, V264T, V266L, S267A, S267Q, S267V, K290D, K290E, K290H, K290L, K290N, K290Q, K290R, K290S, K290Y, P291G, P291Q, P291R, R292I, R292L, E293A, E293D, E293G, E293M, E293Q, E293S, E293T, E294A, E294G, E294P, E294Q, E294R, E294T, E294V, 02951, Q295M, Y296H, S298A, S298R, Y300I, Y300V, Y300W, R301A, R301M, R301P, R301S, V302F, V302L, V302M, V302R, V302S, V303S, V303Y, S3041, V305A, V305F, V3051, V305L, V305R and V305S, wherein the numbering is that of the EU index or equivalent in Kabat.

The present invention also relates to a DNA sequence comprising a gene encoding a variant of a parent polypeptide comprising an Fc fragment, said variant having an increased affinity for the FcRn receptor, and an increased affinity for at least one fragment receptor Fc(FcR) selected from among FcγRI (CD64), FcγRIIIa (CD16a) and FcγRIIa (CD32a) receptors, relative to that of the parent polypeptide, said variant comprising:
(i) the four mutations 334N, 352S, 378V and 397M; and
(ii) at least one mutation selected from among 434Y, 434S, 226G, P228L, P228R, 230S, 230T, 230L, 241L, 264E, 307P, 315D, 330V, 362R, 389T and 389K;
wherein the numbering is that of the EU index or equivalent in Kabat,
said DNA sequence optionally comprising a sequence encoding a signal peptide permitting the secretion of said variant.

In a particular embodiment, said variant further comprising at least one mutation (iii) in the Fc fragment chosen from among Y296W, K290G, V240H, V240I, V240M, V240N, V240S, F241H, F241Y, L242A, L242F, L242G, L242H, L242I, L242K, L242P, L242S, L242T, L242V, F243L, F243S, E258G, E258I, E258R, E258M, E258Q, E258Y, V259C, V259I, V259L, T260A, T260H, T260I, T260M, T260N, T260R, T260S, T260W, V262S, V263T, V264L, V264S, V264T, V266L, S267A, S267Q, S267V, K290D, K290E, K290H, K290L, K290N, K290Q, K290R, K290S, K290Y, P291G, P291Q, P291R, R292I, R292L, E293A, E293D, E293G, E293M, E293Q, E293S, E293T, E294A, E294G, E294P, E294Q, E294R, E294T, E294V, 02951, Q295M, Y296H, S298A, S298R, Y300I, Y300V, Y300W, R301A, R301M, R301P, R301S, V302F, V302L, V302M, V302R, V302S, V303S, V303Y, S304T, V305A, V305F, V3051, V305L, V305R and V305S, wherein the numbering is that of the EU index or equivalent in Kabat.

Alternatively, the polypeptide according to the invention may be produced in cultured mammalian cells. The preferred cells are the YB2/0 rat line, the CHO hamster line, in particular the CHO dhfr- and CHO Lec13 lines, the PER C6™ cells (Crucell), NSO, SP2/0, HeLa, BHK or COS cells, HEK293 cells. Preferably, the CHO hamster line is used.

Thus, the present invention also relates to a process for producing a variant of a parent polypeptide comprising an Fc fragment, said variant having an increased affinity for the FcRn receptor, and an increased affinity for at least one Fc receptor (FcR) selected from FcγRI (CD64), FcγRIIIa (CD16a) and FcγRIIa (CD32a) receptors, relative to that of the parent polypeptide, said variant comprising:
(i) the four mutations 334N, 352S, 378V and 397M; and
(ii) at least one mutation selected from 434Y, 434S, 226G, P228L, P228R, 230S, 230T, 230L, 241L, 264E, 307P, 315D, 330V, 362R, 389T and 389K; and
wherein the numbering is that of the EU index or equivalent in Kabat, said process comprising expressing said variant in mammalian cells in culture.

In a particular embodiment, said variant further comprises at least one mutation (iii) in the Fc fragment chosen from among Y296W, K290G, V240H, V240I, V240M, V240N, V240S, F241H, F241Y, L242A, L242F, L242G, L242H, L242I, L242K, L242P, L242S, L242T, L242V, F243L, F243S, E258G, E258I, E258R, E258M, E258Q, E258Y, V259C, V259I, V259L, T260A, T260H, T260I, T260M, T260N, T260R, T260S, T260W, V262S, V263T, V264L, V264S, V264T, V266L, S267A, S267Q, S267V, K290D, K290E, K290H, K290L, K290N, K290Q, K290R, K290S, K290Y, P291G, P291Q, P291R, R292I, R292L, E293A, E293D, E293G, E293M, E293Q, E293S, E293T, E294A, E294G, E294P, E294Q, E294R, E294T, E294V, 02951, Q295M, Y296H, S298A, S298R, Y300I, Y300V, Y300W, R301A, R301M, R301P, R301S, V302F, V302L, V302M, V302R, V302S, V303S, V303Y, S304T, V305A, V305F, V3051, V305L, V305R and V305S, wherein the numbering is that of the EU index or equivalent in Kabat,

In particular, such a process comprises the following steps:
a) preparing a DNA sequence encoding the variant;
b) introducing the DNA sequence obtained in a) into mammalian cells in culture. The introduction may be carried out transiently or stably (i.e. integration of the DNA sequence obtained in a) into the genome of the cells); and
c) expression of the variant from the cells obtained in b), then
d) optionally, recovery of the variant in the culture medium.

The present invention also relates to a pharmaceutical composition comprising (i) a polypeptide according to the invention, and (ii) at least one pharmaceutically acceptable excipient.

The object of the present invention is also a pharmaceutical composition comprising (i) the variant consisting of an Fc fragment, in particular of IgG1, exhibiting the five mutations N434Y, K334N, P352S, V397M and A378V, wherein the numbering is that of the EU index or equivalent in Kabat, and (ii) at least one pharmaceutically acceptable excipient. Preferably, the composition of the present invention comprises (i) the variant consisting of an Fc fragment, in particular of IgG1, having the six mutations N434Y, K334N, P352S, V397M and A378V, Y296W, the numbering being that of the index EU or equivalent in Kabat, and (ii) at least one pharmaceutically acceptable excipient.

The object of the present invention is also the polypeptide according to the invention or the composition as described above, for its use as a drug.

The object of the present invention is also the use of the variant consisting of an Fc fragment, in particular of IgG1, exhibiting the five mutations N434Y, K334N, P352S, V397M and A378V, wherein the numbering is that of the EU index or equivalent in Kabat. (i.e. variant N434Y/K334N/P352S/V397M/A378V) as a drug. In a particular embodiment, the object of the present invention is also the use of the variant consisting of an Fc fragment, in particular of IgG1, presenting the six mutations N434Y, Y296W, K334N, P352S, V397M, A378V, and Y296W, wherein the numbering is that of the index EU or equivalent in Kabat (i.e. variant N434Y/K334N/P352S/V397M/A378V/Y296W), as a drug.

As indicated above, advantageously, the parent polypeptide—and therefore the polypeptide according to the invention—is an antibody. In this case, the antibody may be directed against an antigen selected from a tumor antigen, a viral antigen, a bacterial antigen, a fungal antigen, a toxin, a membrane or circulating cytokine, and a membrane receptor.

When the antibody is directed against a tumor antigen, its use is particularly suitable in the treatment of cancers. By “cancer” is meant any physiological condition characterized by an abnormal proliferation of cells. Examples of cancers include carcinomas, lymphomas, blastomas, sarcomas (including liposarcomas), neuroendocrine tumors, mesotheliomas, meningiomas, adenocarcinomas, melanomas, leukemias and lymphoid malignancies, wherein this list is not exhaustive.

When the antibody is directed against a viral antigen, its use is particularly useful in the treatment of viral infections. Viral infections include infections caused by HIV, a retrovirus, a Coxsackie virus, smallpox virus, influenza, yellow fever, West Nile, a cytomegalovirus, a rotavirus or hepatitis B or C, wherein this list is not exhaustive.

When the antibody is directed against a toxin, its use is particularly useful in the treatment of bacterial infections, for example infections with tetanus toxin, diphtheria toxin, anthrax toxins Bacillus anthracis, or in the treatment of infections by botulinum toxins, ricin toxins, shigatoxins, wherein this list is not exhaustive.

When the antibody is directed against a cytokine, its use is particularly suitable in the treatment of inflammatory and/or autoimmune diseases. Inflammatory and/or autoimmune diseases include thrombotic thrombocytopenic purpura (ITP), transplant and organ rejection, graft-versus-host disease, rheumatoid arthritis, systemic lupus erythematosus, various types of sclerosis, primary Sjögren's syndrome (or Gougerot-Sjögren's syndrome), autoimmune polyneuropathies such as multiple sclerosis, type I diabetes, autoimmune hepatitis, ankylosing spondylitis, Reiter's syndrome, gout arthritis, celiac disease, Crohn's disease, Hashimoto chronic thyroiditis (hypothyroidism), Adisson's disease, autoimmune hepatitis, Basedow (hyperthyroidism), ulcerative colitis, vasculitis and systemic vasculitis associated with ANCA (anti-cytoplasmic antibodies to neutrophils), autoimmune cytopenia and other hematologic complications in adults and children, such as acute or chronic autoimmune thrombocytopenia, autoimmune haemolytic anemias, haemolytic disease of the newborn (MHN), cold agglutinin disease, autoimmune haemophilia; Goodpasture syndrome, extra-membranous nephropathies, autoimmune bullous skin disorders, refractory myasthenia gravis, mixed cryoglobulinemia, psoriasis, juvenile chronic arthritis, inflammatory myositis, dermatomyositis and systemic autoimmune disorders of the child including antiphospholipid syndrome, connective tissue disease, pulmonary autoimmune inflammation, Guillain-Barré syndrome, chronic inflammatory demyelinating polyradiculoneuropathy (PDCl), autoimmune thyroiditis, mellitis, myasthenia gravis, inflammatory autoimmune disease of the eye, optic neuromyelitis (Devia's disease), scleroderma, pemphigus, insulin resistance diabetes, polymyositis, Biermer's anemia, glomerulonephritis, Wegener's disease, Horton, periarthritis nodosa and Churg and Strauss syndrome, Still's disease, atrophic polychondritis, malaise of Behçcet, monoclonal gammopathy, Wegener's granulomatosis, lupus, ulcerative colitis, psoriatic arthritis, sarcoidosis, collagenous colitis, dermatitis herpetiformis, familial Mediterranean fever, IgA glomerulonephritis, syndrome myasthenic Lambert-Eaton, sympathetic ophthalmia, Fiessinger-Leroy-Reiter syndrome, and uveo-meningoencephalic syndrome.

Other inflammatory diseases are also included, such as acute respiratory distress syndrome (ARDS), acute septic arthritis, adjuvant arthritis, allergic encephalomyelitis, allergic rhinitis, allergic vasculitis, allergy, asthma, atherosclerosis, chronic inflammation due to chronic bacterial or viral infections, chronic obstructive pulmonary disease (COPD), coronary heart disease, encephalitis, inflammatory bowel disease, inflammatory osteolysis, inflammation associated with acute and delayed hypersensitivity reactions, inflammation associated with tumors, peripheral nerve injury or demyelinating diseases, inflammation associated with tissue trauma such as burns and ischemia, inflammation due to meningitis, multiorgan organ failure syndrome (multiple organ dysfunction syndrome, MODS), pulmonary fibrosis, sepsis and septic shock, Stevens-Johnson syndrome, undifferentiated arthritis, and undifferentiated spondyloarthropathies. In a particular embodiment of the invention, the autoimmune disease is idiopathic thrombotic purpura (ITP) and chronic inflammatory demyelinating polyradiculoneuropathy (CIDP).

Preferably, the autoimmune or inflammatory pathology is selected from immunologic thrombocytopenic purpura (also called idiopathic thrombocytopenic purpura, or ITP), optic neuromyelitis or deviant disease (NMO) and multiple sclerosis. Multiple sclerosis and, in particular, experimental autoimmune encephalomyelitis (EAE) is studied thanks to a model.

The sequences described in this application may be summarized as follows:

| SEQ | | |
|---|---|---|
| ID | | |
| NO: | Protein | Sequence |
| 1 | Fc region of human IgG1 | CPPCPAPELLGGPSVFLFPPKPKDTLMISRTPEVTCV |
| | G1m1,17 (residus 226-447 | VVDVSHEDPEVKFNWYVDGVEVHNAKTKPREEQYNST |
| | according to EU index | YRVVSVLTVLHQDWLNGKEYKCKVSNKALPAPIEKTI |
| | or equivalent in Kabat) | SKAKGQPREPQVYTLPPSRDELTKNQVSLTCLVKGFY |
| | without upper hinge | PSDIAVEWESNGQPENNYKTTPPVLDSDGSFFLYSKL |
| | N-terminus region | TVDKSRWQQGNVFSCSVMHEALHNHYTQKSLSLSPGK |
| 2 | Fc region of human IgG2 | CPPCPAPPVAGPSVFLFPPKPKDTLMISRTPEVTCVV |
| | without upper hinge N- | VDVSHEDPEVQFNWYVDGVEVHNAKTKPREEQFNSTF |
| | terminus region | RVVSVLTVVHQDWLNGKEYKCKVSNKGLPAPIEKTIS |
| | | KTKGQPREPQVYTLPPSREEMTKNQVSLTCLVKGFYP |
| | | SDIAVEWESNGQPENNYKTTPPMLDSDGSFFLYSKLT |
| | | VDKSRWQQGNVFSCSVMHEALHNHYTQKSLSLSPGK |
| 3 | Fc region of human IgG3 | CPRCPAPELLGGPSVFLFPPKPKDTLMISRTPEVTCV |
| | without upper hinge N- | VVDVSHEDPEVQFKWYVDGVEVHNAKTKPREEQYNST |
| | terminus region | FRVVSVLTVLHQDWLNGKEYKCKVSNKALPAPIEKTI |
| | | SKTKGQPREPQVYTLPPSREEMTKNQVSLTCLVKGFY |
| | | PSDIAVEWESSGQPENNYNTTPPMLDSDGSFFLYSKL |
| | | TVDKSRWQQGNIFSCSVMHEALHNRFTQKSLSLSPGK |
| 4 | Fc region of human IgG4 | CPSCPAPEFLGGPSVFLFPPKPKDTLMISRTPEVTCV |
| | without upper hinge N- | VVDVSQEDPEVQFNWYVDGVEVHNAKTKPREEQFNST |
| | terminus region | YRVVSVLTVLHQDWLNGKEYKCKVSNKGLPSSIEKTI |
| | | SKAKGQPREPQVYTLPPSQEEMTKNQVSLTCLVKGFY |
| | | PSDIAVEWESNGQPENNYKTTPPVLDSDGSFFLYSRL |
| | | TVDKSRWQEGNVFSCSVMHEALHNHYTQKSLSLSLGK |
| 5 | Fc region of human IgG1 | CPPCPAPELLGGPSVFLFPPKPKDTLMISRTPEVTCV |
| | G1m3 without upper | VVDVSHEDPEVKFNWYVDGVEVHNAKTKPREEQYNST |
| | hinge N-terminus region | YRVVSVLTVLHQDWLNGKEYKCKVSNKALPAPIEKTI |
| | | SKAKGQPREPQVYTLPPSREEMTKNQVSLTCLVKGFY |
| | | PSDIAVEWESNGQPENNYKTTPPVLDSDGSFFLYSKL |
| | | TVDKSRWQQGNVFSCSVMHEALHNHYTQKSLSLSPGK |
| 6 | Fc region of human | EPKSCDKTHTCPPCPAPELLGGPSVFLFPPKPKDTLM |
| | d′IgG1 G1m1,17 with | ISRTPEVTCVVVDVSHEDPEVKFNWYVDGVEVHNAKT |
| | upper hinge N-terminus | KPREEQYNSTYRVVSVLTVLHQDWLNGKEYKCKVSNK |
| | region (residus 216-447 | ALPAPIEKTISKAKGQPREPQVYTLPPSRDELTKNQV |
| | according to EU index or | SLTCLVKGFYPSDIAVEWESNGQPENNYKTTPPVLDS |
| | equivalent in Kabat) | DGSFFLYSKLTVDKSRWQQGNVFSCSVMHEALHNHYT |
| | | QKSLSLSPGK |
| 7 | Fc region of human IgG2 | ERKCCVECPPCPAPPVAGPSVFLFPPKPKDTLMISRT |
| | with upper hinge N- | PEVTCVVVDVSHEDPEVQFNWYVDGVEVHNAKTKPRE |
| | terminus region | EQFNSTFRVVSVLTVVHQDWLNGKEYKCKVSNKGLPA |
| | | PIEKTISKTKGQPREPQVYTLPPSREEMTKNQVSLTC |
| | | LVKGFYPSDIAVEWESNGQPENNYKTTPPMLDSDGSF |
| | | FLYSKLTVDKSRWQQGNVFSCSVMHEALHNHYTQKSL |
| | | SLSPGK |
| 8 | Fc region of human IgG3 | ELKTPLGDTTHTCPRCPEPKSCDTPPPCPRCPEPKSC |
| | with upper hinge N- | DTPPPCPRCPEPKSCDTPPPCPRCPAPELLGGPSVFL |
| | terminus region | FPPKPKDTLMISRTPEVTCVVVDVSHEDPEVQFKWYV |
| | | DGVEVHNAKTKPREEQYNSTFRVVSVLTVLHQDWLNG |
| | | KEYKCKVSNKALPAPIEKTISKTKGQPREPQVYTLPP |
| | | SREEMTKNQVSLTCLVKGFYPSDIAVEWESSGQPENN |
| | | YNTIPPMLDSDGSFFLYSKLTVDKSRWQQGNIFSCSV |
| | | MHEALHNRFTQKSLSLSPGK |
| 9 | Fc region of human IgG4 | ESKYGPPCPSCPAPEFLGGPSVFLFPPKPKDTLMISR |
| | with upper hinge N- | TPEVTCVVVDVSQEDPEVQFNWYVDGVEVHNAKTKPR |
| | terminus region | EEQFNSTYRVVSVLTVLHQDWLNGKEYKCKVSNKGLP |
| | | SSIEKTISKAKGQPREPQVYTLPPSQEEMTKNQVSLT |
| | | CLVKGFYPSDIAVEWESNGQPENNYKTTPPVLDSDGS |
| | | FFLYSRLTVDKSRWQEGNVFSCSVMHEALHNHYTQKS |
| | | LSLSLGK |
| 10 | Fc region of human IgG1 | EPKSCDKTHTCPPCPAPELLGGPSVFLFPPKPKDTLM |
| | G1m3 with upper hinge | ISRTPEVTCVVVDVSHEDPEVKFNWYVDGVEVHNAKT |
| | N-terminus region | KPREEQYNSTYRVVSVLTVLHQDWLNGKEYKCKVSNK |
| | | ALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHYT |
| | | QKSLSLSPGK |
| 11 | Variant Fc A3A-184AY | DKTHTCPPCPAPELLGGPSVFLFPPKPKDTLMISRTP |
| | | EVTCVVVDVSHEDPEVKFNWYVDGVEVHNAKTKPREE |
| | | QYNSTYRVVSVLTVLHQDWLNGKEYKCKVSNKALPAP |
| | | IENTISKAKGQPREPQVYTLSPSRDELTKNQVSLTCL |
| | | VKGFYPSDIVVEWESNGQPENNYKTTPPMLDSDGSFF |
| | | LYSKLTVDKSRWQQGNVFSCSVMHEALHYHYTQKSLS |
| | | LSPGK |
| 12 | Signal peptide | MRWSWIFLLLLSITSANA |
| 13 | Variant Fc A3A-184AY | MRWSWIFLLLLSITSANADKTHTCPPCPAPELLGGPS |
| | with signal peptide | VFLFPPKPKDTLMISRTPEVTCVVVDVSHEDPEVKFN |
| | (i.e. fusion of SEQ ID | WYVDGVEVHNAKTKPREEQYNSTYRVVSVLTVLHQDW |
| | NO: 12 with SEQ ID | LNGKEYKCKVSNKALPAPIENTISKAKGQPREPQVYT |
| | NO: 11) | LSPSRDELTKNQVSLTCLVKGFYPSDIVVEWESNGQP |
| | | ENNYKTTPPMLDSDGSFFLYSKLTVDKSRWQQGNVFS |
| | | CSVMHEALHYHYTQKSLSLSPGK |
| 14 | Fc region of human IgG1 | DKTHTCPPCPAPELLGGPSVFLFPPKPKDTLMISRTP |
| | G1m1,17 with residues | EVTCVVVDVSHEDPEVKFNWYVDGVEVHNAKTKPREE |
| | 221-447 according to EU | QYNSTYRVVSVLTVLHQDWLNGKEYKCKVSNKALPAP |
| | index or equivalent in | IEKTISKAKGQPREPQVYTLPPSRDELTKNQVSLTCL |
| | Kabat | VKGFYPSDIAVEWESNGQPENNYKTTPPVLDSDGSFF |
| | | LYSKLTVDKSRWQQGNVFSCSVMHEALHNHYTQKSLS |
| | | LSPGK |
| 15 | Variant Fc A3A-184EY | DKTHTCPPCPAPELLGGPSVFLFPPKPKDTLMISRTP |
| | | VETCVVVDVSHEDPEVKFNWYVDGVEVHNAKTKPREE |
| | | QWNSTYRVVSVLTVLHQDWLNGKEYKCKVSNKALPAP |
| | | IENTISKAKGQPREPQVYTLSPSRDELTKNQVSLTCL |
| | | VKGFYPSDIVVEWESNGQPENNYKTTPPMLDSDGSFF |
| | | LYSKLTVDKSRWQQGNVFSCSVMHEALHYHYTQKSLS |
| | | LSPGK |
| 16 | Variant Fc A3A-184EY | MRWSWIFLLLLSITSANADKTHTCPPCPAPELLGGPS |
| | with signal peptide | VFLFPPKPKDTLMISRTPEVTCVVVDVSHEDPEVKFN |
| | (i.e. fusion of SEQ ID | WYVDGVEVHNAKTKPREEQWNSTYRVVSVLTVLHQDW |
| | NO: 12 with SEQ ID | LNGKEYKCKVSNKALPAPIENTISKAKGQPREPQVYT |
| | NO: 15) | LSPSRDELTKNQVSLTCLVKGFYPSDIVVEWESNGQP |
| | | ENNYKTTPPMLDSDGSFFLYSKLTVDKSRWQQGNVFS |
| | | CSVMHEALHYHYTQKSLSLSPGK |

The present invention will be better understood upon reading the following examples.

### BRIEF DESCRIPTION OF THE DRAWINGS

FIG. 1: Production of variant A3A-184AY in goat milk and mouse using the vector Bc451

A) The beta casein vector, Bc451, was digested with XhoI.
In the vector Bc451, the NotI-NotI fragment is the prokaryotic fragment. The NotI fragment (15730)-XhoI is the 3′ genomic sequence that contains the polyA signal. The BamHI-XhoI fragment is the promoter region of beta casein.
B) The Sall fragment containing the Fc A3A-184AY variant coding region (i.e. FC3179 A3A-184AY 884 bp) was inserted into the vector, to generate the BC3180 FC A3A-184AY (C) gene construct.
D) The DNA fragment for microinjection was then isolated from the prokaryotic vector. To do this, BC3180 was digested with NotI and NruI. The 16.4 kb fragment, containing the Fc gene (encoding the A3A-184AY variant) under the control of the beta casein promoter, was then purified by gel elution.

FIG. 2: Results of Tests in an Orentive Model of Arthritis Induced by K/B×N Mouse Serum Transfer

The disease was induced by transferring 10 ml of K/B×N mouse serum intravenously on D0 to C57/BI/6J mice. The test molecules were administered once intraperitoneally at D0, 2h before injection of the K/B×N mouse serum.

The clinical score is obtained by summing the four-leg index:

0=normal, 1=swelling of a joint, 2=swelling of more than one joint, and 3=severe swelling of the entire joint (arbitrary units).

FIG. 3: Results of tests in a therapeutic model of arthritis induced by the transfer of K/B×N mouse serum

The disease was induced by transferring 10 ml of K/B×N mouse serum intravenously on D0 to C57/BI/6J mice. The test molecules were administered once intraperitoneally at D0, 72 hours after injection of K/B×N mouse serum (indicated by dotted lines).

The clinical score is obtained by summing the four-leg index:

0=normal, 1=swelling of a joint, 2=swelling of more than one joint, and 3=severe swelling of the entire joint (arbitrary units).

FIG. 4: Test results of binding Fc and IqIV to sanitary cells

IgIV or Fc variants according to the invention labeled with Alexa were incubated at 65 nM (10 μg/ml for Fc in 2% CSF PBS) with target cells for 20 minutes on ice. After 2 washes in 2% CSF, the cells were suspended in 500 ml Isoflow prior to flow cytometric analysis.

The results are as follows:

A) B cells labeled with anti-CD19 (“% positive B cells”);
B) NK cells labeled with anti-CD56 (“% positive NK cells”);
C) monocytes labeled with anti-CD14, in the presence of IgIV (“% positive cells+IgIV”);
D) CD16+monocytes labeled with anti-CD14 and with the anti-CD16 3G8 antibody, in the presence of IgIV (“% positive cells+IgIV”);
E) Neutrophils labeled with anti-CD15, in the presence of IgIV (“% positive cells+IgIV”);
F) NK cells labeled with anti-CD56, in the presence of IgG or Fc WT (“% cell positive”).

FIG. 5: Results of ADCC tests, activation of Jurkat CD64 and CDC cells

A) Inhibition of activation of Jurkat CD64 cells:
Raji cells (50 ml at 5×10⁶ cells/nil) were mixed with Rituxan (50 ml to 2m9/ml), Jurkat cells expressing human CD64 (Jurkat-H-CD64) (25 ml at 5×10⁶ cells/ml), PMA (50 ml to 40 ng/ml), then incubated with IgIV or the variant according to the invention (RFC A3A-184AY) at 1950 nM.

After a night of incubation, the plates were centrifuged (125 g for 1 minute), and IL2 contained in the supernatant was evaluated by ELISA.

The results were expressed as a percentage with respect to IgIV, according to the following formula: (IL-2 IgIV/IL-2 of the sample)×100.

### B) Inhibition of ADCC:

Effector cells (mononuclear cells) (25 ml at 8×10⁷ cells/nil) and Rh-positive RBCs (25 ml at 4×10⁷ cells/ml final) were incubated with different concentrations (0 to 75 ng/ml) of anti-Rh-antibody D, with an Effector/Target ratio of 2/1. After 16 hours of incubation, lysis was estimated by quantifying the hemoglobin released into the supernatant using a specific substrate (DAF).

The results are expressed as a percentage of specific lysis as a function of the amount of antibody. Inhibition of ADCC was induced by IgG or Fc variant according to the invention (RFC A3A-184AY) added at 33 nM.

The results are expressed in percent, wherein 100% and 0% are the values obtained with IgIV at 650 nM and 0 nM respectively according to the following formula:

[(ADCC with 33 nM sample−ADCC without IVIg)/(ADCC with IgIV at 33 nM—ADCC without IVIg)×100].

### C) Inhibition Activity of the CDC:

Raji cells were incubated for 30 minutes with a final concentration of 50 ng/ml of rituximab. A solution of young rabbit serum diluted 1/10 and previously incubated with the variant Fc according to the invention (rFc A3A-184AY) or IgIV (vol/vol) for 1 h at 37° C. was added. After 1 hour of incubation at 37° C., the plates were centrifuged (125 g for 1 minute) and the CDC was estimated by measuring the intracellular LDH released in the culture medium. The results were expressed as percent inhibition and compared to IgG and negative control (Fc without Fc function, i.e. rFc neg), 100% corresponding to a complete inhibition of lytic activity and 0% to the control value obtained without Fc or IgIV.

FIG. 6: Results of the Cell Binding Tests

IgIV, Fc-Rec (wild-type Fc), Fc MST-HN or Fc variants according to the invention (A3A-184AY CHO, A3A-184EY CHO) labeled with Alexa-Fluor® were incubated at 65 nM (10 μg/ml) for Fc in 2% CSF (Colony Stimulating Factor) PBS with target cells for 20 minutes on ice. After 2 washes in 2% CSF PBS, the cells were suspended in 500 μl of Isoflow before flow cytometric analysis The tests are performed on the following target cells:
Natural Killer (NK) cells labeled with anti-CD56;
Monocytes labeled with anti-CD14;
CD16+monocytes labeled with anti-CD14 and anti-CD16 3G8 antibody;
Neutrophils labeled with anti-CD15.

FIG. 7: Results of tests in an in vivo model of idiopathic thrombocytopenic purpura (ITP)

The disease was induced in mice expressing humanized FcRn by injecting an anti-platelet antibody 6A6-hlgG1 (0.3 pg/g body weight) intravenously to deplete platelets, also called thrombocytes, from mice. Negative Control (“CTL PBS”), IgIV (1000 mg/kg), Fc-Rec (Fc-wild) fragment (380 and 750 mg/kg), Fc MST-HN fragment (190 mg/kg) and the variant of the invention Fc A3A-184AY CHO (190 mg/kg and 380 mg/kg), were administered intraperitoneally 2 hours before platelet depletion. Platelet count was determined with an Advia Hematology system (Bayer). The number of platelets before the injection of antibodies was set at 100%.

### DESCRIPTION OF THE PREFERRED EMBODIMENTS

### Example 1: Preparation of Variants (Mutated Fc Fragments) According to the Invention Produced in the Milk of Transgenic Animals and Characterization of Said Variants

I. Materials and Methods

Principle:

An Fc fragment according to the invention may be produced in the milk of transgenic animals, by placing the coding sequence of the Fc fragment in a milk-specific expression vector. The vector may be introduced into the genome of a transgenic mouse or goat by microinjection. Following the screening and identification of an animal with the transgene, the females are reproduced. Following the parturition, milking the females allows recovery of their milk, in which the Fc could be secreted following the expression of the specific promoter of the milk.

Protein Sequence of Fc Variant A3A-184AY (K334N/P352S/A378V/V397M/N434Y):

(SEQ ID NO: 11)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIENTISKAKGQPREPQVYTLSPSRDELTKNQVSLTCLVK
GFYPSDIVVEWESNGQPENNYKTTPPMLDSDGSFFLYSKLTVDKSRWQQG
NVFSCSVMHEALHYHYTQKSLSLSPGK

A signal peptide (MRWSWIFLLLLSITSANA, SEQ ID NO: 12) is bound to the N-terminus of the protein sequence, so as to obtain the sequence SEQ ID NO: 13. It allows the secretion of the protein in milk, once expressed.

### Optimization of the Nucleotide Sequence:

The nucleotide sequence has been optimized for expression in the goat mammary gland. For this, the sequence was optimized for the Bos taurus species by the algorithm of a synthetic gene provider (such as GeneArt).

### Expression Vector:

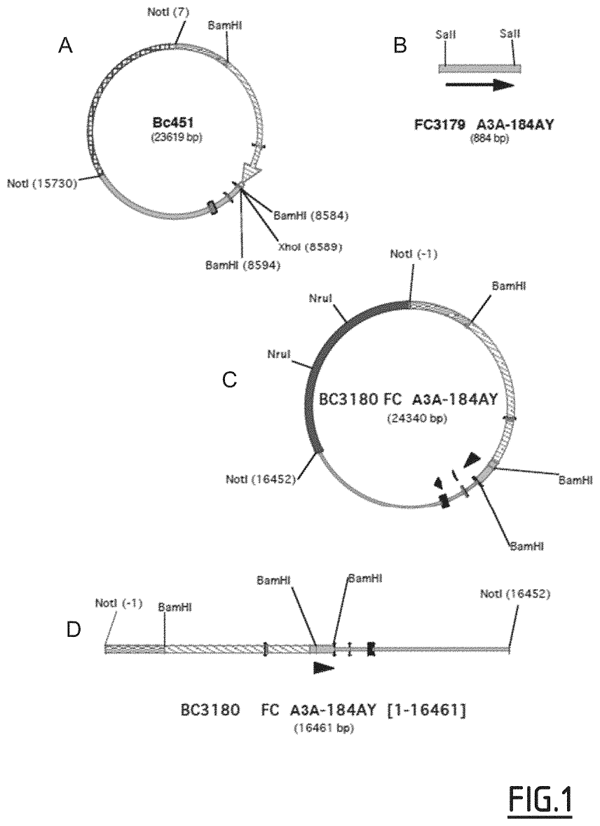

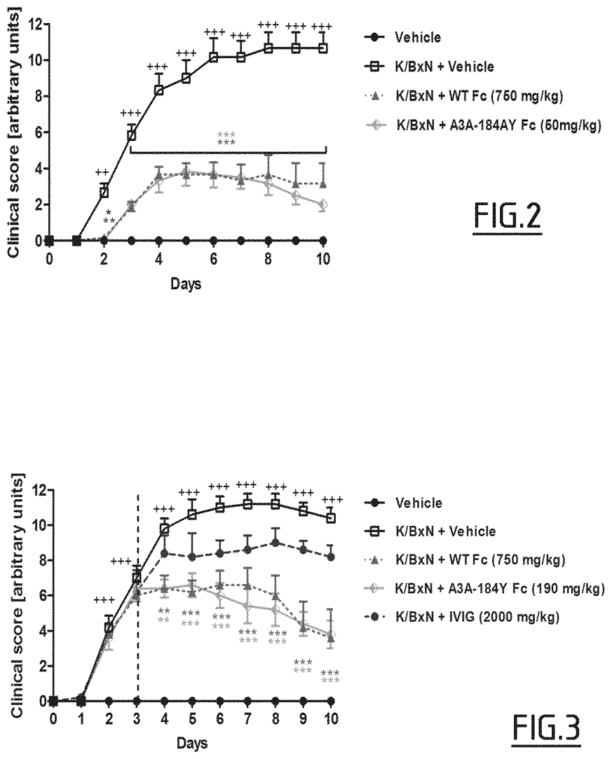

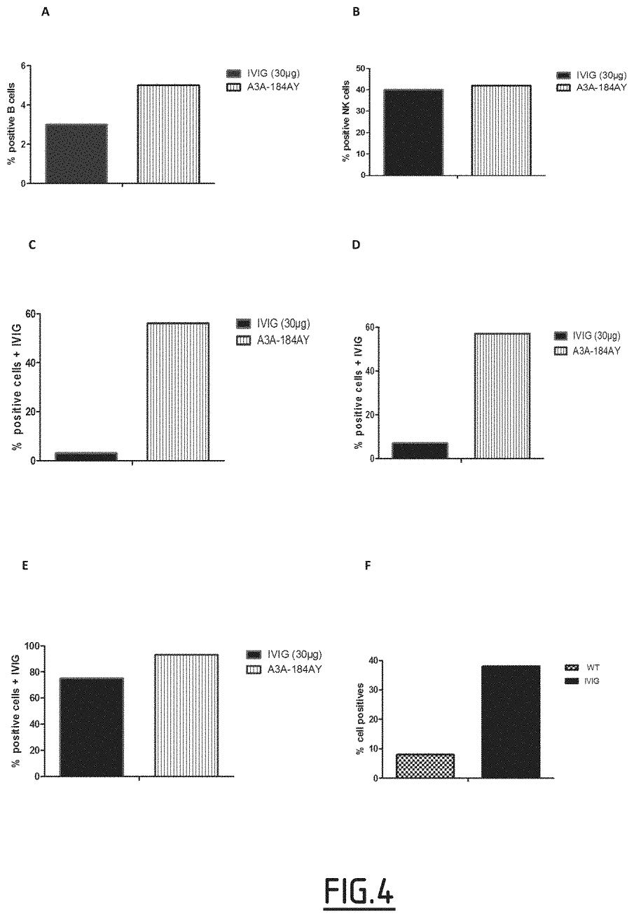

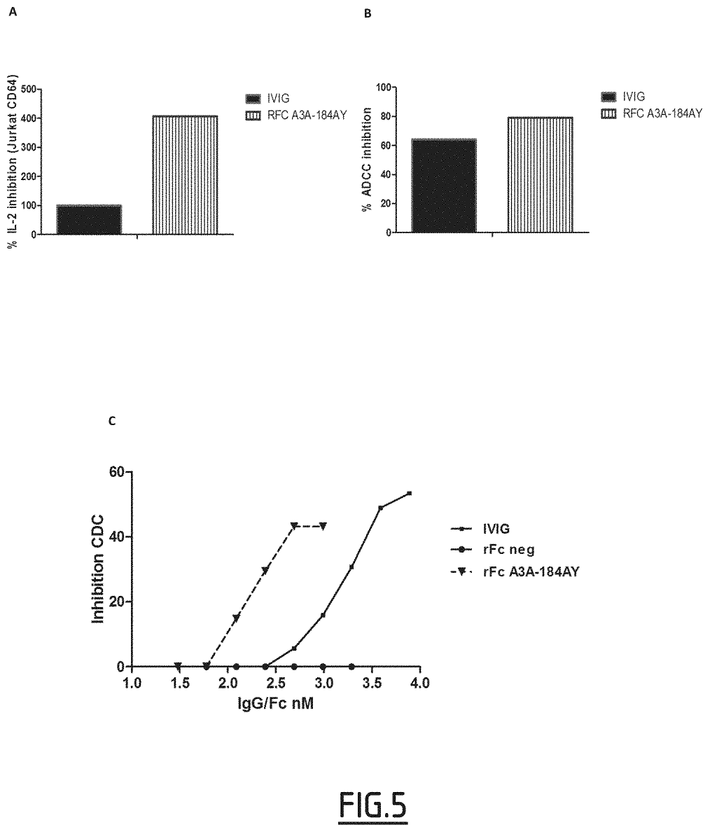

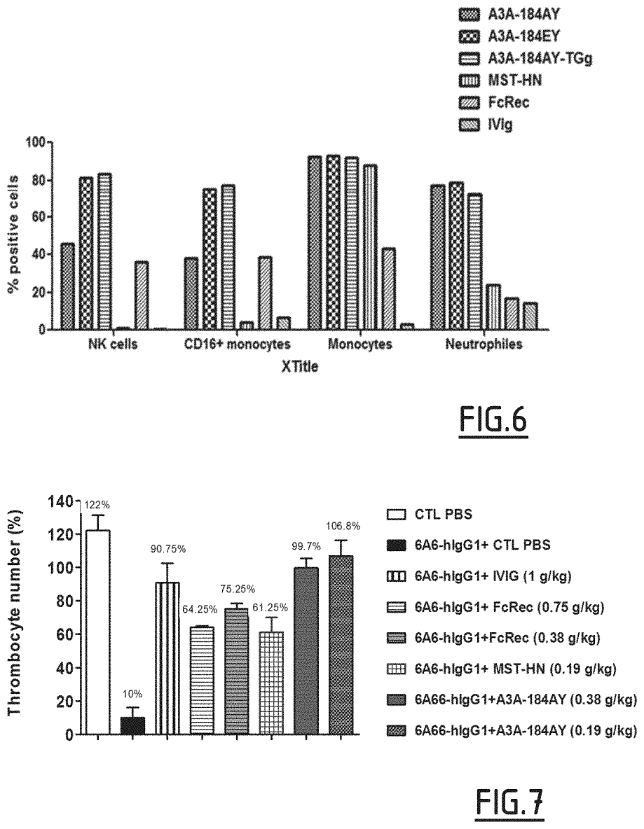

The goat beta casein expression vector (Bc451) was used for the production of the A3A-184AY variant in mouse and goat milk (see FIG. 1).

The beta casein vector, Bc451, was digested with XhoI (FIG. 1A). The Sall fragment containing the Fc A3A-184AY variant coding region was inserted to generate the BC3180 FC A3A-184AY gene construct (FIGS. 1B and 1C).

The DNA fragment for microinjection was then isolated from the prokaryotic vector.

BC3180 was digested with NotI and NruI (FIG. 1D). The released 16.4 kb fragment containing the Fc gene under the control of the beta casein promoter was then purified by gel elution. This DNA was then used in the microinjection stage.

### Production in the Mouse:

The DNA fragment was inserted by microinjection into preimplantation mouse embryos. The embryos were then implanted in pseudopregnant females. The offspring that were born were screened for the presence of the transgene by PCR analysis.

### Expression in Goats:

The DNA fragment prepared for microinjection may also be used for the production of the Fc variant A3A-184AY in goat's milk.

### Example 2: Preparation of Variants (Mutated Fc Fragments) According to the Invention, Produced in HEK Cells and Characterization of Said Variants

I. Materials and Methods for Production

Each mutation of interest in the Fc fragment of sequence SEQ ID NO: 14 was inserted by overlap PCR using two sets of primers adapted to integrate the targeted mutation(s) with the codon(s) encoding the desired amino acid. Advantageously, when the mutations to be inserted are close to the Fc sequence, they are added via the same oligonucleotide. The fragments thus obtained by PCR were combined and the resulting fragment was amplified by PCR using standard protocols. The PCR product was purified on 1% (w/v) agarose gel, digested with the appropriate restriction enzymes and cloned.

The recombinant Fc fragment was produced by transient transfection (by lipofection) in HEK293 cells (293-F cells, InvitroGen freestyle) in F17 medium supplemented with L-glutamine using the pCEP4 vector. After 8 days of culture, the supernatant is clarified by centrifugation and filtered through a 0.2 μm filter. Fragment Fc is then purified on Hi-Trap protein A, and elution is effected with 25 mM citrate buffer pH=3.0, neutralized and dialyzed in PBS prior to filtration sterilization (0.2 μm).

II. Octet® Binding Tests (BLI Technology “Bio-Layer Interferometry”, Device: Byte RED96, Fortebio, PaII)

Protocols:

Human FcRn Binding (hFcRn):

The biotinylated hFcRn receptor is immobilized on Streptavidin Biosensors, diluted to 0.7 μg/ml in run buffer (0.1 M phosphate buffer, 150 mM NaCl, 0.05% Tween 20, pH6). The variants according to the invention, WT and IgIV, were tested at 200, 100, 50, 25, 12.5, 6.25, 3.125 and 0 nM in run buffer (200 nM=10 μg/ml for Fc).

Design of the Test:

Baseline 1×120 s in run buffer

Loading 300 s: the receiver is loaded on the biosensors

Baseline 2×60 s in run buffer

Association 60 s: samples (Fc or IVIg) are added to the biosensors loaded in hFcRn

Dissociation 30 s in run buffer

Regeneration 120 s in regeneration buffer (0.1 M phosphate buffer, 150 mM NaCl, 0.05% Tween 20, pH 7.8).

Results Interpretation:

The association and dissociation curves (first 10 s) are used to calculate the kinetic constants of association (kon) and dissociation (koff) using a 1/1 association model. KD (nM) is then calculated (kon/koff).

Link to the hCD16aV and hCD32aH Receivers:

The hCD16aV (R&D System) or hCD32aH (PX therapeutics) HisTag receptor is immobilized on anti-Penta-HIS Biosensors (HIS 1K), diluted to 1 μg/ml in kinetic buffer (PaII). The Fc variants according to the invention, WT and IgIV, were tested at 1000, 500, 250, 125, 62.5, 31.25, 15 and 0 nM in kinetic buffer.

Loading Before Each Sample

Design of the test: All the stages are realized in kinetic buffer (PaII)

Baseline 1×60 s

Loading 400 s

Baseline 2×60 s

Association 60 s

Dissociation 30 s

Regeneration 5 s in regeneration buffer (Glycine 10 mM pH 1.5/Neutralization: PBS).

Results Interpretation:

The association and dissociation curves (first 5 s) are used to calculate the kinetic constants of association (kon) and dissociation (koff) using a 1/1 association model. KD (nM) is then calculated (kon/koff).

Results:

The results are shown in Table 1 below:

TABLE 1

| Molecule | hCD16aV | SD | hFcRn | SD | hCD32aH | SD |
|---|---|---|---|---|---|---|
| IVIg | 653.8 | 4.0 | 34.4 | 1.94 | 438.2 | 114.3 |
| Fc-WT (HEK) | 504.3 | 75.0 | 36.5 | 8.2 | 659.3 | 203.1 |
| A3A-184AY | 132.0 | 14.1 | 7.8 | 0 | 313.0 | 29.7 |
| (HEK) | | | | | | |

SD = standard deviation

The results show that the variant Fc A3A 184AY (HEK) according to the invention exhibits both an increased affinity for the hFcRn receptor, and an increased affinity for the FcγRIIIa (CD16a) and FcγRIIa (CD32a) receptors, and this compared to Fc parent not mutated (Fc-WT) but also compared to IVIG.

III. Model-Based Arthritis Assays Induced by K/B×N Mouse Serum Transfer

Protocol:

The K/B×N model was generated by crossing the transgenic mice for the KRN T cell receptor to the NOD mouse strain. K/B×N F1 mice spontaneously develop a disease at 3 to 5 weeks of age and share many clinical features with human rheumatoid arthritis.

The disease was induced by transferring 10 ml of K/BxN mouse serum intravenously on D0 to C57/BI/6J mice. The molecules tested were administered once intraperitoneally at D0, 2h before or 72 hours after the injection of K/BxN mouse serum.

Mice were monitored daily for signs and symptoms of arthritis to assess incidence and severity by adding the four-leg index:

0=normal, 1=swelling of a joint, 2=swelling of more than one joint, and 3=severe swelling of the entire joint.

Results:

Mice given K/BxN serum developed arthritis in the joint. The disease was characterized by an increase in ankle size, leading to an increase in the clinical score. These mice showed a significant increase in clinical score and ankle thickness compared to control mice treated with saline.

1—Preventive Model:

Administered 2 h before the K/BxN mouse serum injection, treatment with 750 mg/kg of wild-type Fc (Fc WT) fragment significantly reduced the clinical score compared to the serum group of K/BxN mice.

Treatment with the Fc variant A3A-184AY (HEK) according to the invention significantly reduced the clinical score in a manner similar to the Fc WT fragment, but at a dose 15 times lower (50 mg/kg) (FIG. 2).

2—Therapeutic Model:

72 hours after the injection of K/B×N mouse serum, the IgG administered at 2 g/kg did not significantly reduce the clinical score compared to the group treated with K/B×N mouse serum.

However, treatment with the Fc WT fragment at 750 mg/kg (molecular dose equivalent to 2 g/kg IVIG) significantly reduced the clinical score compared to the group treated with K/B×N mouse serum. In addition, treatment with the variant Fc A3A-184AY (HEK) according to the invention significantly reduced the clinical score similarly to the Fc-WT fragment, but at a dose 4-fold lower (190 mg/kg) (FIG. 3).

IV. In Vitro Cell Tests

Protocols:

Evaluation of the Binding of Fc and Ig IV Fragments to Blood Cells:

IgIV or Fc variants according to the invention labeled with Alexa were incubated at 65 nM (10 μg/ml for Fc in 2% CSF PBS) with target cells for 20 minutes on ice. After 2 washes in 2% CSF, the cells were suspended in 500 ml Isoflow prior to flow cytometric analysis. B cells, NK cells, monocytes and neutrophils were specifically labeled with anti-CD19, anti-CD56, anti-CD14 and anti-CD15 respectively. The FcγRIII receptor (CD16) was demonstrated using the anti-CD16 3G8 antibody.

Inhibition of ADCC:

To mimic the lysis of red blood cells observed in idiopathic thrombocytopenic purpura (ITP), involving the autoantibodies of the patient with ITP, an effector cell-mediated red cell lysis in the presence of an anti-Rhesus D (RhD) monoclonal anti-body was conducted, and the ability of different amounts of polyvalent immunoglobulins (IVIg) or mutated or non-mutated recombinant Fc fragments, to inhibit this lysis, for example by competition with anti-RhD for fixation of Fc receptors on the surface of the effector cell, were evaluated.

The cytotoxicity of anti-RhD antibodies has been studied by the technique of ADCC. Briefly, effector cells (mononuclear cells) (25 to 8×10⁷ cells/nil) and Rh-positive red cells (25 to 4×10⁷ cells/ml final) were incubated with different concentrations (0 to 75 ng/ml) of anti-RhD antibodies, with an Effector/Target ratio of 2/1. After 16 hours of incubation, lysis was estimated by quantifying the hemoglobin released into the supernatant using a specific substrate (DAF).

The results are expressed as a percentage of specific lysis as a function of the amount of antibody. The inhibition of ADCC induced by IgIV or the Fc variant according to the invention (RFC A3A-184AY) added to 33 nM was evaluated.

The results are expressed in percent, wherein 100% and 0% are the values obtained with IgIV at 650 nM and 0 nM respectively, according to the following formula:

[(ADCC with 33 nM sample−ADCC without IVIg)/(ADCC with IgIV at 33 nM−ADCC without IVIg)×100].

Inhibition of Activation of Jurkat CD64 Cells:

This test estimates the ability of the Fc variants according to the invention or IVIG (total IgG), to inhibit the secretion of IL2 by Jurkat cells expressing human CD64 (Jurkat-H-CD64) induced by the Raji cell line with Rituxan.

Briefly, Raji cells (50 ml at 5×10⁶ cells/nil) were mixed with Rituxan (50 ml at 2 mg/ml), Jurkat H-CD64 cells (25 ml at 5×10⁶ cells/ml, a phorbol ester (PMA, 50 ml at 40 ng/ml), then incubated with the IgIV or the Fc variant according to the invention at 1950 nM.

After a night of incubation, the plates were centrifuged (125 g for 1 minute) and NL2 contained in the supernatant was evaluated by ELISA.

The results were expressed as a percentage with respect to IgIV, according to the following formula:

(IL-2 IgIV/IL-2 of the sample)×100.

Inhibitory Activity of the CDC:

This assay estimates the ability of the Fc variant according to the invention or IVIG to inhibit rituximab-mediated CDC activity on the Raji cell line in the presence of rabbit serum as a source of complement. Briefly, Raji cells were incubated for 30 minutes with a final concentration of 50 ng/ml of rituximab. A solution of young rabbit serum diluted 1/10 and previously incubated with the variant according to the invention or IgIV (vol/vol) for 1 h at 37° C., was added. After 1 hour of incubation at 37° C., the plates were centrifuged (125 g for 1 minute) and the CDC was estimated by measuring the intracellular LDH released in the culture medium.

The results were expressed as percentage inhibition and compared to IVIG and negative control (Fc without Fc function), 100% corresponding to a complete inhibition of lytic activity and 0% to the control value obtained without Fc or IVIG.

Results:

The results are shown in FIGS. 4 and 5.

As shown in FIG. 5, the Fc variant according to the invention (A3A-184AY (HEK)) has a better inhibition of the activity of the Jurkat cells expressing CD64, of the ADCC and of the CDC, in comparison with the IVIg. These results show that a variant according to the invention such as A3A-184AY may be effective for the treatment of pathologies involving patient autoantibodies, in particular by blocking Fc receptors on the effector cells of the patient (see FIG. 4).

### Example 3: Preparation of Variants (Mutated Fc Fragments) According to the Invention, Produced in CHO Cells

The recombinant Fc fragment may be obtained from SEQ ID NO: 14 in the same manner as that described in Example 2. This mutated Fc fragment may be produced by transfection into CHO—S cells with the aid of lipofection such as Freestyle Max Reagent (Thermofisher) using a vector optimized for expression in this cell line. The CHO—S cells are cultured in CD FortiCHO medium+8 mM Glutamine, under conditions agitated at 135 rpm in a controlled atmosphere (8% CO₂) at 37° C. On the day before the day of transfection, the cells are seeded at a density of 6.10⁵ cells/ml.

On the day of transfection, the linearized DNA (50 μg) and 50 μl of transfection agent (TA) are pre-incubated separately in Opti-Pro SFM medium and then mixed and incubated for 20 minutes to allow the formation of the DNA/AT complex. The whole is then added to a cell preparation of 1.10⁶ cells/ml in a volume of 30 ml. After 48 hours of incubation, transfection agents are added (Neomycin 1 g/L and Methotrexate 200 nM) to the cells. The cell density and viability are determined every 3-4 days and the culture volumes adapted to maintain a cell density greater than 6.10⁵ cells/ml. When the viability is greater than 90%, the stable pools obtained are saved by cryostatic congelation and productions in agitated conditions are carried out in “Fed-batch” mode for 10 days with an addition of 4 g/l or 6 g/l of glucose during production. At the end of production, the cells and the supernatant are separated by centrifugation. The cells are removed and the supernatant is harvested, concentrated and filtered at 0.22 μm.

The Fc fragment is then purified by affinity chromatography on a protein A resin (HiTrap protein A, GE Healthcare). After capture on the balanced resin PBS buffer, the Fc fragment is eluted with 25 mM citrate buffer pH=3.0, followed by rapid pH neutralization with 1M Tris and then dialysed in PBS buffer before sterilization by filtration (0.2 pm).

### Example 4: Binding Tests of FcRn, CD16aH, CD16aV, CD64 and CD32a Variants Produced in CHO Cells and in Transgenic Goat Milk

Fc receptor binding assays are performed with the following molecules:
Variants of the invention A3A-184AY CHO (K334N/P352S/A378V/V397M/N434Y), A3A-184EY_CHO (Y296W/K334N/P352S/A378V/V397M/N434Y) produced in CHO cells according to the process given in example 3, A3A-184AY_TGg produced in the transgenic goat according to the process described in Example 1;
The Fc MST-HN fragment containing the mutations M252Y/S254T/T256E/H433K/N434F, described in the literature as having an optimized binding only to the FcRn receptor (Ulrichts et al, JCI, 2018) was produced in HEK-293 cells. (293-F cells, InvitroGen freestyle);
A wild-type Fc Fc-WT or Fc-Rec fragment obtained by digesting with papain an IgG1 produced in transgenic goat milk;
IVIG

Human FcRn Binding (hFcRn):

FcRn binding is studied by competitive assay using A488 labeled Rituxan (Rituxan-A488) and Jurkat cells expressing the FcRn receptor (Jurkat-FcRn).

The Jurkat-FcRn cells are seeded in a 96-well plate (V bottom) at a concentration of 2.10⁵ cells per well. The cells are then incubated for 20 minutes at 4° C. with the test molecules diluted in buffer at the following final concentrations: 167 μg/ml; 83 μg/ml; 42 μg/ml; 21 μg/ml; 10 μg/ml; 5 μg/ml; 3 μg/ml; 1 μg/ml; 0 μg/ml, and simultaneously with 25 μg/ml Rituxan-A488.

The cells are then washed by adding 100 μl of PBS at pH 6 and centrifuged at 1700 rpm for 3 minutes at 4° C. The supernatant is then removed and 300 μl of cold PBS is added at pH 6.

The binding of Rituxan-A488 to FcRn expressed by Jurkat-FcRn cells is evaluated by flow cytometry. The mean fluorescence intensity (MFI) observed are expressed as a percentage, wherein 100% is the value obtained with Rituxan-A488 alone, and 0% the value in the absence of Rituxan-A488. The molecular concentrations required to induce 50% inhibition of Rituxan-A488 binding to FcRn of Jurkat-FcRn cells are calculated using “Prism Software”.

The results are shown in Table 2 below.

TABLE 2

| | A3A- | A3A- | A3A- | MST- | | |
|---|---|---|---|---|---|---|
| | 184AY_CHO | 184EY_CHO | 184AY_TGg | HN | Fc-WT | IVIG |
| Inhibition of | 13 | 15 | 12 | 14 | 476 | 1356 |
| binding to FcRn | | | | | | |
| (IC 50%, nM) | | | | | | |

The results show that the Fc A3A-184AY CHO, Fc A3A-184EY CHO and A3A-184AY-TGg variants show increased Rituxan-A488 binding inhibition (×100 compared to IVIG). The variants of the invention show an FcRn binding affinity equivalent to that observed with the Fc MST-HN fragment described in the literature as optimized only for FcRn (Ulrichts et al, JCI, 2018).

Binding to hCD64 and hCD16aH, hCD16aV, hCD32aH, hCD32aR Receptors:

Binding to Human CD64 (hCD64)

Human CD64 binding is studied by competitive assay using Rituxan-A488 and Jurkat cells expressing the CD64 receptor (Jurkat-CD64).

Jurkat-CD64 cells are seeded in a 96-well plate (V-bottom) at a concentration of 2.10⁵ cells per well. The cells are then incubated for 20 minutes at 4° C. with the test molecules diluted in the buffer with the final concentrations: 167 μg/ml; 83 μg/ml; 42 μg/ml; 21 μg/ml; 10 μg/ml; 5 μg/ml; 3 μg/ml; 1 μg/ml; 0 μg/ml, and simultaneously with 25 μg/ml Rituxan-A488.

The cells are then washed by adding 1 μl of PBS at pH 6 and centrifuged at 1700 rpm for 3 minutes at 4° C. The supernatant is then removed and 300 μl of cold PBS is added at pH 6.

The binding of Rituxan-A488 to CD64 expressed by Jurkat-CD64 cells is evaluated by flow cytometry. The mean fluorescence intensities (MFI) observed are expressed as a percentage, wherein 100% is the value obtained with Rituxan-A488 alone, and 0% is the value in the absence of rituxan-A488. The molecular concentrations required to induce 50% inhibition of Rituxan-A488 binding to CD64 of Jurkat-CD64 cells are calculated using “Prism Software”.

Binding to CD32aH and CD32aR Human CD32 receptor binding is studied by competitive assay using Rituxan-A488 and HEK cells transfected with CD32aH and CD32aR (HEK-CD32) receptors.

The HEK-CD32 cells are seeded in a 96-well plate (V bottom) at a concentration of 2.10⁵ cells per well. The cells are then incubated for 20 minutes at 4° C. with the test molecules diluted in buffer at the following final concentrations: 333 μg/ml; 167 μg/ml, 83 μg/ml; 42 μg/ml; 21 μg/ml; 10 μg/ml; 5 μg/ml; 3 μg/ml; 1 μg/ml; 0 μg/ml, and simultaneously with 30 μg/ml Rituxan-A488.

The cells are then washed by adding 100 μl of PBS at pH 6 and centrifuged at 1700 rpm for 3 minutes at 4° C. The supernatant is then removed and 300 μl of cold PBS is added at pH 6.

The binding of Rituxan-A488 to CD32aH and CD32aR expressed by HEK-CD32 cells is evaluated by flow cytometry. The mean fluorescence intensities (MFI) observed are expressed as a percentage, wherein 100% is the value obtained with the Rituxan-A488 alone, and 0% is the value in the absence of Rituxan-A488. The molecular concentrations required to induce 50% inhibition of Rituxan-A488 binding to CD32aH and CD32aR of HEK-CD32 cells are calculated using “Prism Software”.

Binding to hCD16aH

The binding to human CD16aH is studied by competitive assay using a murine anti-CD16 3G8 antibody labeled with phycoerythrin (3G8-PE) and Jurkat cells transfected with the human CD16aH receptor (Jurkat-CD16aH).

The Jurkat-CD16aH cells are seeded in a 96-well plate (V bottom) at a concentration of 2.10⁵ cells per well. The cells are then incubated for 20 minutes at 4° C. with the test molecules diluted in buffer at the following final concentrations: 83 μg/ml; 42 μg/ml; 21 μg/ml; 10 μg/ml; 5 μg/ml; 3 μg/ml; 1 μg/ml; 0 μg/ml, and simultaneously with 0.5 μg/ml mAb 3G8-PE.

The cells are then washed by adding 1 μl of PBS at pH 6 and centrifuged at 1700 rpm for 3 minutes at 4° C. The supernatant is then removed and 300 μl of cold PBS is added at pH 6.

The binding of mAb 3G8-PE to CD16aH expressed by Jurkat-CD16aH cells is evaluated by flow cytometry. The average fluorescence intensities (MFI) observed are expressed as a percentage, wherein 100% is the value obtained with the mAb 3G8-PE alone, and 0% is the value in the absence of mAb 3G8-PE. The molecular concentrations required to induce 50% inhibition of mAb 3G8-PE binding to CD16aH of Jurkat-CD16aH cells, are calculated using “Prism Software”.

The results are shown in Table 3 below.

TABLE 3

| | A3A- | A3A- | A3A- | MST- | | |
|---|---|---|---|---|---|---|
| | 184AY_CHO | 184EY_CHO | 184AY_TGg | HN | Fc-WT | IVIG |
| Inhibition of | 262 | 123 | 105 | >2170 | 282 | 1684 |
| binding to the | | | | | | |
| CD16a-F (IC | | | | | | |
| 50%, nM) | | | | | | |
| Inhibition of | 135 | 147 | 170 | >2170 | >2170 | 671 |
| binding to the | | | | | | |
| CD32a-H (IC | | | | | | |
| 50%, nM) | | | | | | |
| Inhibition of | 176 | 132 | Not | >2170 | >2170 | 1308 |
| binding to the | | | determined | | | |
| CD32a-R (IC | | | | | | |
| 50%, nM) | | | | | | |
| Inhibition of | 57 | 55 | 59 | >2170 | >2170 | 761 |
| binding to the | | | | | | |
| CD32b (IC | | | | | | |
| 50%, nM) | | | | | | |
| Inhibition of | 84 | 70 | 87 | 494 | 176 | 880 |
| binding to the | | | | | | |
| CD64 (IC | | | | | | |
| 50%, nM) | | | | | | |

The results show that the A3A-184AY CHO Fc, A3A-184EY CHO Fc and A3A-184AY_TGg variants have an increased affinity for the FcγRIIIa (CD16a), FcγRI (CD64) and FcγRIIa (CD32a) receptors, compared to the Fc non mutated (Fc-WT) but also compared to IVIG.

The mutants of the invention show a very increased affinity for FcγRIIIa (CD16a), FcγRI (CD64) and FcγRIIa (CD32a) receptors compared to MST-HN.

Binding to Human CD16aV:

HisTag hCD16aV (R&D System) receptor is immobilized on anti-Penta-HIS Biosensors (HIS 1K), diluted to 1 μg/ml in kinetic buffer (PaII). The molecules were tested at concentrations of 1000, 500, 250, 125, 62.5, 31, 25, 15 and 0 nM in kinetic buffer.

Loading Before Each Sample

Design of the Test: All the Steps are Realized in Kinetic Buffer (PaII)

Baseline 1×60 s

Loading 400 s

Baseline 2×60 s

Association 60 s

Dissociation 30 s

Regeneration 5 s in regeneration buffer (Glycine 10 mM pH 1.5/Neutralization: PBS).

Results Interpretation:

The association and dissociation curves (first 5 s) are used to calculate the kinetic constants of association (kon) and dissociation (koff) using a 1/1 association model. KD (nM) is then calculated (kon/koff).

The results are shown in Table 4 below.

TABLE 4

| Molecule | KD hCD16aV (nM) | SD |
|---|---|---|
| A3A-184AY_CHO | 80.3 | 18.1 |
| A3A-184EY_CHO | 59.3 | 7.7 |
| A3A-184AY_TGg | 51.2 | 10.7 |
| MST-HN | 268.2 | 83.6 |
| Fc-WT | 314.1 | 72.7 |
| IVIG | 339.0 | 103.9 |

SD: standard deviation

The results show that the Fc A3A-184AY CHO, Fc A3A-184EY CHO and A3A-184AY_TGg variants show a binding increase for the human FcγRIIIa-V receptor (CD16a-V), and this compared to the non-mutated Fc (Fc-WT) but also compared to IgM and Fc fragment MST-HN containing M252Y/S254T/T256E/H433K/N434F mutations.

### Example 5: ADCC Inhibition and Jurkat Cell Activation Tests of Variants Produced in CHO Cells and in Transgenic Goat Milk

ADCC inhibition and Jurkat cell activation tests are performed with the following molecules:
Variants of the invention A3A-184AY_CHO (K334N/P352S/A378V/V397M/N434Y), A3A-184EY_CHO (Y296W/K334N/P352S/A378V/V397M/N434Y) produced in CHO cells according to the process given in Example 3,
The Fc MST-HN fragment containing the M252Y/S254T/T256E/H433K/N434F mutations, described in the literature as having a binding optimized only to the FcRn receptor (Ulrichts et al, JCI, 2018) was produced in HEK-293 cells (293-F cells, Freestyle InvitroGen),
A wild-type Fc “Fc-Rec” or “Fc-WT” fragment, obtained by digesting with papain an IgG1 produced in transgenic goat's milk,
IgIV

ADCC Inhibition Test:

To mimic the lysis of red blood cells observed in idiopathic thrombocytopenic purpura (ITP), involving the autoantibodies of the patient with ITP, an effector cell-mediated red cell lysis in the presence of a Rhesus D (RhD) anti-human monoclonal antibody was conducted, and the ability of different amounts of polyvalent immunoglobulins (IgMV) or mutated or non-mutated recombinant Fc fragments, to inhibit this lysis, for example by competition with anti-RhD for fixation Fc receptors on the surface of the effector cells, were evaluated.

The cytotoxicity of anti-RhD antibodies has been studied by the technique of ADCC. Briefly, effector cells (mononuclear cells) (25 to 8×10⁷ cells/nil) and Rh-positive red cells (25 to 4×10⁷ cells/ml final) were incubated with different concentrations (0 to 75 ng/ml) of anti-RhD antibodies, with an Effector/Target ratio of 2/1. After 16 hours of incubation, lysis was estimated by quantifying the hemoglobin released into the supernatant using a specific substrate (DAF).

The results are expressed as a percentage of specific lysis as a function of the amount of antibody. The inhibition of ADCC is induced by the molecules tested (IgM, MST-HN, Fc-WT A3A-184AY CHO, A3A-184EY CHO) at concentrations of 500, 50, 5, 0.5 μg/ml. for MST-HN, Fc-WT A3A-184AY_CHO, A3A-184EY_CHO and 1500, 150, 15, 1.5 μg/ml for IgIV. The molecule concentrations to induce 25% or 50% inhibition were calculated with “Prism Software”.

The results are shown in Table 5 below.

TABLE 5

| | A3A- | A3A- | MST- | | |
|---|---|---|---|---|---|
| | 184AY_CHO | 184EY_CHO | HN | Fc-WT | IVIg |
| Inhibition of the lysis of | 13.5 | 7.6 | 190.2 | 82 | 59.6 |
| the red blood cells | | | | | |
| medited by the anti-D | | | | | |
| AD1 (IC 25%, nM) | | | | | |
| Inhibition of the lysis of | 97 | 56 | 441 | 1500 | 351 |
| the red blood cells | | | | | |
| medited by the anti-D | | | | | |
| AD1 (IC 50%, nM) | | | | | |

The results show that the Fc variants, A3A-184AY CHO and A3A-184EY CHO, show an inhibition of lysis of red blood cells by an increased anti-Rhesus D antibody compared to non-mutated Fc (Fc-WT) but also compared with IVIG.

In addition, the inhibition of A3A-184AY CHO or A3A-184EY CHO is greatly increased compared to the Fc fragment, MST-HN, containing the M252Y/S254T/T256E/H433K/N434F mutations.

Inhibition of Activation of Jurkat CD64 Cells:

This test estimates the ability of the Fc variants according to the invention or IVIG (total IgG) to inhibit the secretion of IL2 by Jurkat cells expressing human CD64 (Jurkat-H-CD64) induced by the Raji cell line with Rituxan.

Briefly, Raji cells (50 ml at 5×10⁶ cells/nil) were mixed with Rituxan (50 ml at 2 mg/ml), Jurkat H-CD64 cells (25 ml at 5×10⁶), a phorbol ester (PMA, 50 ml at 40 ng/ml), then incubated with the IGVI or Fc variant according to the invention at 1950 nM.

After a night of incubation, the plates were centrifuged (125 g for 1 minute) and NL2 contained in the supernatant was evaluated by ELISA.

Inhibition of IL2 secretion was induced by IVIG, Fc-WT, MST-HN or Fc variants according to the invention (A3A-184AY CHO or A3A-184EY CHO) added at 50 and 100 μg/ml. for Fc-WT, MST-HN fragments or Fc variants according to the invention (A3A-184AY CHO or A3A-184EY CHO), and 150 and 300 μg/ml for IGVI.

The concentrations of the molecule to induce 25% or 50% inhibition were calculated with “Prism Software”.

The results are shown in Table 6 below.

TABLE 6

| | A3A- | A3A- | MST- | | |
|---|---|---|---|---|---|
| | 184AY_CHO | 184EY_CHO | HN | Fc-WT | IVIG |
| Inhibition of the | 448 | 442 | 1455 | 926 | 1106 |
| secretion of IL-2 of the | | | | | |
| Jurkat cells transfected | | | | | |
| with CD64 (IC 25%, nM) | | | | | |
| Inhibition of the | 600 | 600 | <1950 | <1950 | <1950 |
| secretion of IL-2 of the | | | | | |
| Jurkat cells transfected | | | | | |
| with CD64 (IC 50%, nM) | | | | | |

The results show that the A3A-184AY-CHO and A3A-184EY-CHO Fc variants show increased inhibition of IL2 secretion compared to non-mutated Fc (Fc-WT) but also compared to IVIG.

In addition, the inhibition of RFC A3A-184AY CHO or A3A-184EY CHO is greatly increased compared to the MST-HN Fc fragment containing the M252Y/S254T/T256E/H433K/N434F mutations.

### Example 6: Tests of Binding Fc Variant to Blood Cells

The blood cell binding tests are performed with the following molecules:
Variants of the invention A3A-184AY CHO (K334N/P352S/A378V/V397M/N434Y), A3A-184EY_CHO (Y296W/K334N/P352S/A378V/V397M/N434Y) produced in CHO cells according to the process given in example 3, A3A-184AY_TGg produced in the transgenic goat according to the process described in Example 1,
The fragment Fc MST-HN containing the mutations M252Y/S254T/T256E/H433K/N434F, described in the literature as having an optimized binding only to the FcRn receptor (Ulrichts et al, JCI, 2018), was produced in HEK-293 cells (293-F cells, Freestyle InvitroGen),
A wild-type Fc “Fc-Rec” or “Fc-WT” fragment, obtained by digesting with papain an IgG1 produced in transgenic goat's milk,
IgIV

The molecules labeled with the Alexa Fluor® marker (highly fluorescent protein marker) were incubated at 65 nM (10 μg/ml for Fc in 2% CSF PBS) with target cells for 20 minutes on ice.

After 2 washes in 2% CSF, the cells were suspended in 500 ml Isoflow prior to flow cytometric analysis. The tests are performed on the following cells:
Natural Killer (NK) cells labeled with anti-CD56 (“% positive NK cells”);
Monocytes labeled with anti-CD14 (“% positive cells”);
CD16+ monocytes labeled with anti-CD14 and with the anti-CD16 3G8 antibody (“% positive cells”);
Neutrophils labeled with anti-CD15 (“% positive cells”)

The FcγRIII receptor (CD16) was demonstrated using the anti-CD16 3G8 antibody.

The results show that the variants Fc A3A-184AY CHO, A3A-184EY CHO and A3A-184AY_TGg, whatever the mode of production, offer increased binding compared to the non-mutated Fc (Fc-Rec), but also compared to the IgIV. In addition, the binding of A3A-184AY or A3A-184EY is greatly increased compared to the MST-HN fragment for NK cells, CD16+ monocytes and neutrophils (see FIG. 6).

### Example 7: In Vivo Model Tests of Idiopathic Thrombocytopenic Purpura (ITP)

The disease was induced in mice expressing a humanized FcRn (mFcRn−/−hFcRnTg 276 heterozygous B6 gene background (The Jackson Laboratory) by injecting an anti-platelet antibody 6A6-hIgG1 (0.3 pg/g body weight) intravenously to deplete the platelets of the mice. A blood test is made (number of thrombocytes) 24 hours before the injection of 6A6-hIgG1, 4h after the induction of the disease. The IgIV (1000 mg/kg), Fc-Rec (380 and 750 mg/kg), Fc MST-HN (190 mg/kg) and Fc A3A-184AY CHO (190 mg/kg and 380 mg/kg), were administered intraperitoneally 2 hours before platelet depletion.

Platelet count was determined with an Advia Hematology system (Bayer). The number of platelets before the injection of antibodies was set at 100%.

The anti-platelet antibody 6A6-hIgG1 (0.3 μg/g) makes it possible to deplete 90% of the platelets.

The administration of drug candidates 2 hours before depletion of platelets can restore (FIG. 7):
100% platelets for A3A 184AY CHO at a dose of 380 mg/kg;
106% platelets for A3A-184AY CHO at a dose of 190 mg/kg;
90% platelets for IgIV at a dose of 1000 mg/kg;
64% platelets for Fc-WT at a dose of 750 g/kg;
75% platelets for Fc-WT at a dose of 380 mg/kg;
61% of the platelets for the MST-HN variant at a dose of 190 mg/kg.

1. Variant of a parent polypeptide comprising an Fc fragment, said variant having an increased affinity for the FcRn receptor, and an increased affinity for at least one Fc receptor (FcR) selected from the FcγRI (CD64), FcγRIIIa (CD16a) and FcγRIIa (CD32a), relative to that of the parent polypeptide, comprising:
(i) the four mutations 334N, 352S, 378V and 397M; and
(ii) at least one mutation selected from 434Y, 434S, 226G, P228L, P228R, 230S, 230T, 230L, 241L, 264E, 307P, 315D, 330V, 362R, 389T and 389K;
wherein the numbering is that of the EU index or equivalent in Kabat.

2. The variant according to claim 1, further comprising at least one mutation (iii) in the Fc fragment chosen from among Y296W, K290G, V240H, V240I, V240M, V240N, V240S, F241H, F241Y, L242A, L242F, L242G, L242H, L242I, L242K, L242P, L242S, L242T, L242V, F243L, F243S, E258G, E258I, E258R, E258M, E258Q, E258Y, V259C, V259I, V259L, T260A, T260H, T260I, T260M, T260N, T260R, T260S, T260W, V262S, V263T, V264L, V264S, V264T, V266L, S267A, S267Q, S267V, K290D, K290E, K290H, K290L, K290N, K290Q, K290R, K290S, K290Y, P291G, P291Q, P291R, R292I, R292L, E293A, E293D, E293G, E293M, E293Q, E293S, E293T, E294A, E294G, E294P, E294Q, E294R, E294T, E294V Q295I, Q295M, Y296H, S298A, S298R, Y300I, Y300V, Y300W, R301A, R301M, R301P, R301S, V302F, V302L, V302M, V302R, V302S, V303S, V303Y, S3041, V305A, V305F, V3051, V305L, V305R and V305S,
wherein the numbering is that of the EU index or equivalent in Kabat,

3. The variant according to claim 1, comprising:
(i) the four mutations 334N, 352S, 378V and 397M;
(ii) at least one mutation selected from 434Y, 434S, 226G, P228L, P228R, 230S, 230T, 230L, 241L, 264E, 307P, 315D, 330V, 362R, 389T and 389K; and
(iii) at least one mutation selected from K290G and Y296W,
wherein the numbering is that of the EU index or equivalent in Kabat.

4. The variant according to claim 1, having an increased affinity for the FcRn receptor, relative to that of the parent polypeptide, of a ratio at least equal to 2.

5. The variant according to claim 1, having an increased affinity for at least one Fc receptor (FcR) selected from FcγRI receptors (CD64), FcγRIIIa (CD16a) and FcγRIIα (CD32a), relative to that of the parent polypeptide, of a ratio at least equal to 2.

6. The variant according to claim 1, wherein the variant is produced in mammary epithelial cells of transgenic non-human mammals.

7. The variant according to claim 1, wherein the variant is produced in transgenic non human animals.

8. The variant according to claim 7, wherein the transgenic non-human animal is a transgenic goat.

9. The variant according to claim 1, wherein the variant the parent polypeptide comprises a parent Fc fragment which is a human Fc fragment.

10. The variant according to claim 1, wherein the variant is selected from an isolated Fc fragment, a sequence derived from an isolated Fc fragment, an antibody, an antibody fragment comprising an Fc fragment, and a fusion protein comprising an Fc fragment.

11. The variant according to claim 1, directed against an antigen selected from a tumor antigen, a viral antigen, a bacterial antigen, a fungal antigen, a toxin, a membrane or circulating cytokine, a membrane receptor.

12. A method for treating a patient in need thereof, comprising administering an effective amount of the variant according to claim 1 to said patient.

13. A method for treating an autoimmune or inflammatory pathology, comprising administering an effective amount of the variant according to claim 1 to a patient in need thereof.

14. Pharmaceutical composition comprising a variant according to claim 1, and at least one pharmaceutically acceptable excipient.

15. Process of producing a variant of a parent polypeptide comprising an Fc fragment, said variant having increased affinity for the FcRn receptor, and increased affinity for at least one Fc receptor (FcR) selected from FcγRI receptors (CD64), FcγRIIIa (CD16a) and FcγRIIa (CD32a), relative to that of the parent polypeptide, comprising:
(i) the four mutations 334N, 352S, 378V and 397M; and
(ii) at least one mutation selected from 434Y, 434S, 226G, P228L, P228R, 230S, 230T, 230L, 241L, 264E, 307P, 315D, 330V, 362R, 389T and 389K;
wherein the numbering is that of the EU index or equivalent in Kabat, said process comprising expressing said variant in mammary epithelial cells of transgenic non-human mammals, or said process comprising expressing said variant in mammalian cells in culture.

16. The process for producing a variant of a parent polypeptide comprising an Fc fragment according to claim 15, wherein said variant further comprises at least one mutation (iii) in the Fc fragment chosen from among Y296W, K290G, V240H, V240I, V240M, V240N, V240S, F241H, F241Y, L242A, L242F, L242G, L242H, L242I, L242K, L242P, L242S, L242T, L242V, F243L, F243S, E258G, E258I, E258R, E258M, E258Q, E258Y, V259C, V259I, V259L, T260A, T260H, T260I, T260M, T260N, T260R, T260S, T260W, V262S, V263T, V264L, V264S, V264T, V266L, S267A, S267Q, S267V, K290D, K290E, K290H, K290L, K290N, K290Q, K290R, K290S, K290Y, P291G, P291Q, P291R, R292I, R292L, E293A, E293D, E293G, E293M, E293Q, E293S, E293T, E294A, E294G, E294P, E294Q, E294R, E294T, E294V, Q295I, Q295M, Y296H, S298A, S298R, Y300I, Y300V, Y300W, R301A, R301M, R301P, R301S, V302F, V302L, V302M, V302R, V302S, V303S, V303Y, S304T, V305A, V305F, V3051, V305L, V305R and V305S,
wherein the numbering is that of the EU index or equivalent in Kabat.

17. The process of producing a variant of a polypeptide comprising an Fc fragment according to claim 15, comprising the steps of:
a) preparing a DNA sequence comprising a sequence encoding the variant, a sequence encoding a mammalian casein promoter or a mammalian whey promoter, and a sequence encoding a signal peptide permitting the secretion of said variant;
b) introducing the DNA sequence obtained in a) into a non-human mammalian embryo, to obtain a transgenic non-human mammal expressing the variant encoded by said DNA sequence obtained in a) in the mammary gland; and
c) recovery of the variant in the milk produced by the transgenic nonhuman mammal obtained in b).

18. The process for producing a variant of a polypeptide comprising an Fc fragment according to claim 15, wherein the transgenic non-human mammal is selected from cattle, pigs, goats, sheep and rodents.

19. The process for producing a variant of a polypeptide comprising an Fc fragment according to claim 15, comprising the steps of:
a) preparing a DNA sequence encoding the variant;
b) introducing the DNA sequence obtained in a) into mammalian cells in transient or stable culture;
c) expression of the variant from the cells obtained in b), and
d) recovering the variant in the culture medium.

20. DNA sequence comprising a gene encoding a variant of a parent polypeptide comprising an Fc fragment, said variant having increased affinity for the FcRn receptor, and an increased affinity for at least one Fc receptor (FcR) selected from the receptors FcγRI (CD64), FcγRII1a (CD16α) and FcγRI1a (CD32α), relative to that of the parent polypeptide, wherein said variant comprises:
(i) the four mutations 334N, 352S, 378V and 397M; and
(ii) at least one mutation selected from 434Y, 434S, 226G, P228L, P228R, 230S, 230T, 230L, 241L, 264E, 307P, 315D, 330V, 362R, 389T and 389K;
wherein the numbering is that of the EU index or equivalent in Kabat.

21. DNA sequence comprising a gene encoding a variant of a parent polypeptide comprising an Fc fragment according to claim 20, said variant further comprising at least one mutation (iii) in the Fc fragment selected from Y296W, K290G, V240H, V240I, V240M, V240N, V240S, F241H, F241Y, L242A, L242F, L242G, L242H, L242I, L242K, L242P, L242S, L242T, L242V, F243L, F243S, E258G, E258I, E258R, E258M, E258Q, E258Y, V259C, V259I, V259L, T260A, T260H, T260I, T260M, T260N, T260R, T260S, T260W, V262S, V263T, V264L, V264S, V264T, V266L, S267A, S267Q, S267V, K290D, K290E, K290H, K290L, K290N, K290Q, K290R, K290S, K290Y, P291G, P291Q, P291R, R292I, R292L, E293A, E293D, E293G, E293M, E293Q, E293S, E293T, E294A, E294G, E294P, E294Q, E294R, E294T, E294V, Q295I, Q295M, Y296H, S298A, S298R, Y300I, Y300V, Y300W, R301A, R301M, R301P, R301S, V302F, V302L, V302M, V302R, V302S, V303S, V303Y, S3041, V305A, V305F, V3051, V305L, V305R and V305S,
wherein the numbering is that of the EU index or equivalent in Kabat.


SEQUENCE LISTING (16 sequences)

SEQ ID NO: 1 - protein, 222 aa, Artificial Sequence
  Fc region of the human IgG1 G1m1,17 (residues 226-447 according to the EU index or equivalent in Kabat) without the N-terminus upper hinge region
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 221 aa, Artificial Sequence
  Fc region of the human IgG2 without the N-terminus upper hinge region
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 222 aa, Artificial Sequence
  Fc region of the human IgG3 without the N-terminus upper hinge region
CPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 222 aa, Artificial Sequence
  Fc region of the human IgG4 without the N-terminus upper hinge region
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 222 aa, Artificial Sequence
  Fc region of the human IgG1 G1m3 without the N-terminus upper hinge region
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Fc region of the human IgG1 G1m1,17 with the N-terminus upper hinge region (residues 216-447 according to the EU index or equivalent in Kabat)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, Artificial Sequence
  Fc region of the human IgG2 with the N-terminus upper hinge region
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 279 aa, Artificial Sequence
  Fc region of the human IgG3 with the N-terminus upper hinge region
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 229 aa, Artificial Sequence
  Fc region of the human IgG4 with the N-terminus upper hinge region
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 232 aa, Artificial Sequence
  Fc region of the human IgG1 G1m3 with the N-terminus upper hinge region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 227 aa, Artificial Sequence
  Variant Fc A3A_184AY
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIENTISKAKGQPREPQVYTLSPSRDELTKNQVSLTCLVKGFYPSDIVVEWESNGQPENNYKTTPPMLDSDGSFFLYSKLTVDKSRWQQGNVFSCSVMHEALHYHYTQKSLSLSPGK

SEQ ID NO: 12 - protein, 18 aa, Artificial Sequence
  signal peptide
MRWSWIFLLLLSITSANA

SEQ ID NO: 13 - protein, 245 aa, Artificial Sequence
  Variant Fc A3A_184AY with signal peptide
MRWSWIFLLLLSITSANADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIENTISKAKGQPREPQVYTLSPSRDELTKNQVSLTCLVKGFYPSDIVVEWESNGQPENNYKTTPPMLDSDGSFFLYSKLTVDKSRWQQGNVFSCSVMHEALHYHYTQKSLSLSPGK

SEQ ID NO: 14 - protein, 227 aa, Artificial Sequence
  Fc region of the human IgG1 G1m1,17 with residues 221-447
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 227 aa, Artificial Sequence
  Variant Fc A3A-184EY
DKTHTCPPCPAPELLGGPSVFLFPPKPKDTLMISRTPEVTCVVVDVSHEDPEVKFNWYVDGVEVHNAKTKPREEQWNSTYRVVSVLTVLHQDWLNGKEYKCKVSNKALPAPIENTISKAKGQPREPQVYTLSPSRDELTKNQVSLTCLVKGFYPSDIVVEWESNGQPENNYKTTPPMLDSDGSFFLYSKLTVDKSRWQQGNVFSCSVMHEALHYHYTQKSLSLSPGK

SEQ ID NO: 16 - protein, 245 aa, Artificial Sequence
  Variant Fc A3A-184EY with signal peptide
MRWSWIFLLLLSITSANADKTHTCPPCPAPELLGGPSVFLFPPKPKDTLMISRTPEVTCVVVDVSHEDPEVKFNWYVDGVEVHNAKTKPREEQWNSTYRVVSVLTVLHQDWLNGKEYKCKVSNKALPAPIENTISKAKGQPREPQVYTLSPSRDELTKNQVSLTCLVKGFYPSDIVVEWESNGQPENNYKTTPPMLDSDGSFFLYSKLTVDKSRWQQGNVFSCSVMHEALHYHYTQKSLSLSPGK